(19) United States
(12) Patent Application Publication
(10) Pub. No.: US 2024/0293229 A1
(43) Pub. Date: Sep. 5, 2024

(54) EXPANDABLE SHEATH FOR AN IMPLANT

(71) Applicant: MiRus LLC, Marietta, GA (US)

(72) Inventors: Angela Rothfuss, Marietta, GA (US); Darren DeMedici, Marietta, GA (US); Jay Yadav, Atlanta, GA (US); Noah Roth, Marietta, GA (US)

(21) Appl. No.: 18/663,735

(22) Filed: May 14, 2024

Related U.S. Application Data
(63) Continuation-in-part of application No. 17/873,808, filed on Jul. 26, 2022.
(63) Continuation-in-part of application No. 29/881,979, filed on Jan. 9, 2023.
(63) Continuation-in-part of application No. 29/882,840, filed on Jan. 19, 2023.
(63) Continuation-in-part of application No. 29/883,021, filed on Jan. 23, 2023.
(63) Continuation-in-part of application No. 29/870,899, filed on Feb. 8, 2023.
(60) Provisional application No. 63/534,705, filed on Aug. 25, 2023.
(60) Provisional application No. 63/225,803, filed on Jul. 26, 2021.
(60) Provisional application No. 63/388,782, filed on Jul. 13, 2022.

(51) Int. Cl. A61F 2/24

(52) U.S. Cl. CPC A61F 2/243

(57) ABSTRACT

The present disclosure relates to a sheath that is usable with a medical device, particularly to a sheath that is usable with a medical device and which sheath is expandable in the regions where the medical device is positioned in the sheath, and more particularly to a sheath that is usable with a medical device and which sheath is expandable in the regions where the medical device is positioned in the sheath and which sheath reforms to its same or similar size and shape after the medical device has passed through a portion or all of the sheath. The sheath facilitates in the insertion of a medical device into the body passageway of a patient.

### REFERENCED APPLICATIONS

The present disclosure claims priority on U.S. Provisional Patent Application Ser. No. 63/534,705 filed Aug. 25, 2023, which is fully incorporated herein.

The present disclosure is a continuation-in-part of U.S. patent application Ser. No. 17/873,808 filed Jul. 26, 2022, which in turn claims priority on U.S. Provisional Patent Application Ser. No. 63/225,803 filed Jul. 26, 2021, and U.S. Provisional Patent Application Ser. No. 63/388,782 filed Jul. 13, 2022, which are fully incorporated herein.

The present disclosure is a continuation-in-part of U.S. patent application Ser. No. 29/881,979 filed Jan. 9, 2023, which is fully incorporated herein.

The present disclosure is a continuation-in-part of U.S. patent application Ser. No. 29/882,840 filed Jan. 19, 2023, which is fully incorporated herein.

The present disclosure is a continuation-in-part of U.S. patent application Ser. No. 29/883,021 filed Jan. 23, 2023, which is fully incorporated herein.

The present disclosure is a continuation-in-part of U.S. patent application Ser. No. 29/870,899 filed Feb. 8, 2023, which is fully incorporated herein.

### FIELD OF DISCLOSURE

The present disclosure relates generally to a sheath that is usable with a medical device, particularly to a sheath that is usable with a medical device and which sheath is expandable in at least the region where the medical device is positioned in the sheath, and more particularly to a sheath that is usable with a medical device and which sheath is expandable in at least the region where the medical device is positioned in the sheath and which sheath reforms to its same or similar size and shape after the medical device has passed through a portion of all of the sheath. The sheath can be used to protect the body passageway of a patient and/or incision into a body passageway as a medical device is inserted into and/or through the body passageway and to a treatment site. The sheath can be used with catheter arrangements for stent procedures, angioplasty procedures, embolism procedures, repairing and/or replacing heart valves, etc.

### BACKGROUND

Many modern-day medical devices such as expandable balloons, stents and heart valves are inserted through blood vessels until the medical device reaches a treatment site, and thereafter the medical device is expanded at the treatment site to repair treatment site and/or to secure the medical device at the treatment site. Many medical devices are formed or include metal materials that can include sharp or abrasive edges or ends. Also, the size of the medical device as it passes into and through a body passageway can cause trauma to the body passageway as a) the medical device and delivery device are inserted into an incision in a patient and into the body passageway, b) the medical device passes through the body passageway to the treatment site, and c) the delivery device is removed from the body passageway after delivery of the medical device at the treatment site.

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

A sheath can be used to introduce a delivery apparatus into a patient's vasculature. A sheath can be used to minimize damage to a body passageway as the delivery apparatus is inserted through an incision and into a body passageway (e.g., the femoral artery, etc.). A sheath generally has an elongated sleeve that is inserted into the vasculature and a housing that allows a delivery apparatus to be placed in fluid communication with the vasculature with minimal blood loss. A conventional sheath typically requires a tubular loader to be inserted through the seals in the housing to provide an unobstructed path through the housing for a valve mounted on a balloon catheter. A conventional loader extends from the proximal end of the expandable sheath. Non-limiting prior art sheaths are disclosed in U.S. Pat. Nos. 8,690,936; 8,790,387; 9,192,751; 9,301,840; 9,301,841; 9,956,376; 9,585,743; 9,801,619; 9,907,931; 9,987,134; 10,098,734; 10,327,896; 10,391,279; 10,499,895; 10,517,720; 10,524,905; 10,524,906; 10,524,907; 10,548,631; 10,639,152; 10,661,049; 10,792,150; 10,799,685; 10,912,919; 10,918,829; 10,980,569; 11,026,719; 11,045,317, 11,129,959; 11,213,318; 11,273,062; 11,517,432; 11,596,766; 11,648,115; 11,786,695; US 2016/0106562; US 2021/00156612; US 2022/0401216; US 2023/0054952; US 2023/0056245; US 2023/0149674; US 2023/0172715; US 2023/0263991; US 2023/0270546; WO 2023/014551; WO 2023/091413; and WO 2023/167811; all of which are fully incorporated herein by reference.

Conventional methods of accessing a vessel prior to introducing the delivery system include dilating the vessel using multiple dilators or sheaths that progressively increase in diameter.

Radially expanding intravascular sheaths tend to have complex mechanisms, such as ratcheting mechanisms that maintain the shaft or sheath in an expanded configuration once a device with a larger diameter than the expandable sheath's original diameter is introduced.

The delivery and/or removal of medical device to or from the body passageway of a patient can expose the patient to risk. Furthermore, accessing a body passageway and inserting the medical device into a body passageway while damaging the medical device and/or severely damaging the body passageway continues to be a challenge due in part to the relatively large profile of the delivery system that can cause longitudinal and radial tearing of the blood vessel during insertion into the blood vessel.

Accordingly, there remains a need for an improved sheath for endovascular systems used for implanting stents, valves, and other medical devices.

### SUMMARY OF THE DISCLOSURE

The present disclosure relates generally to a sheath that is usable with a medical device, particularly to a sheath that is usable with a medical device and which sheath is expandable in at least the regions where the medical device is positioned in the sheath, and more particularly to a sheath that is usable with a medical device and which sheath is expandable in at least the regions where the medical device is positioned in the sheath and which sheath reforms to its same or similar size and shape after the medical device has passed through a portion or all of the sheath. The sheath can be used to protect the body passageway of a patient and/or incision into a body passageway as a medical device is inserted into and/or through the body passageway and to a treatment site. The sheath that can be used with catheter arrangements for stent procedures, angioplasty procedures, embolism procedures, repairing and/or replacing heart valves, etc.

In one non-limiting aspect of the present disclosure, there is provided an expandable sheath can be used to reduce or minimize trauma to a body passageway (e.g., blood vessel, duct, etc.) by allowing for temporary expansion of a portion of the expandable sheath to accommodate at least a portion of the delivery system and/or medical device, followed by a return to the same or similar original diameter or cross-sectional area once the delivery system and/or medical device has partially or fully passes through the expandable sheath. In one non-limiting embodiment, the expandable sheath is configured to expand from a first outer diameter or first outer cross-sectional area to a second outer diameter or second outer cross-sectional area as a medical device (e.g., TAVR, heart valve, stent, angioplasty device, etc.) is moved through an interior passageway in the expandable sheath, and thereafter the expandable sheath partially or fully returns to the first outer diameter or first outer cross-sectional area once the medical device has passed partially or fully through the expandable sheath. The first outer diameter or first outer cross-sectional area is less than the second outer diameter or second outer cross-sectional area. In another non-limiting embodiment, the expandable sheath can be configured such that the outer diameter or outer cross-sectional area of the expandable sheath and the inner diameter or inner cross-sectional area of the interior passageway progressively expands and contracts as the medical device is moved through the expandable sheath. In such a configuration, only a portion of the expandable sheath where the medical device is located and/or near the location of the medical device (e.g., 0.1-20 mm within the location of the medical device and all values and ranges therein) in the interior passageway of the expandable sheath expands from the first outer diameter or first outer cross-sectional area to a second outer diameter or second outer cross-sectional area. In the region of the expandable sheath that is located forwardly and/or rearwardly of where the medical device is located in the expandable sheath, the expandable sheath may have an outer diameter or outer cross-sectional area that is the same or similar or greater than the first outer diameter or first outer cross-sectional area, but is less than the second outer diameter or second outer cross-sectional area of the expandable sheath wherein the medical device is located in the expandable sheath. As such, the expandable sheath can be configured to progressively increase in outer diameter or cross-sectional area and then progressively decrease in outer diameter or cross-sectional area as the medical device moves through the interior passageway of the expandable sheath. In another non-limiting embodiment, the expandable sheath can be configured such that the outer diameter or outer cross-sectional area of the expandable sheath and the inner diameter or inner cross-sectional area of the interior passageway expands along the longitudinal length of the expandable sheath as the medical device moves through the expandable sheath, and then partially or fully returns to the first outer diameter or first outer cross-sectional area and first inner diameter or first inner cross-sectional area of the interior passageway once the medical device partially or fully passes through the expandable sheath. The first inner diameter or first inner cross-sectional area is less than the second inner diameter or second inner cross-sectional area. In another non-limiting embodiment, the expandable sheath is partially or fully formed of a shape memory material that facilitates in causing the expandable sheath to partially or fully return to the first outer diameter or first outer cross-sectional area once the medical device has partially or fully passed through the expandable sheath.

In another and/or alternative non-limiting aspect of the present disclosure, the expandable sheath is partially or fully formed of a polymer material. Non-limiting polymers that can be used include polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks, or combinations thereof. In one non-limiting embodiment, the expandable sheath is formed of 30-100 wt. % (and all values and ranges therebetween) of polymer. In one non-limiting specific configuration, 30-85 wt. % of the expandable sheath is formed of polymer.

In another and/or alternative non-limiting aspect of the present disclosure, the expandable sheath is partially or fully formed of a polymer material and wherein the expandable sheath includes at least two polymer layers, wherein the first polymer layer constitutes 5-50 wt. % of the total polymer in the expandable sheath, and the second polymer layer constitutes 5-50 wt. % of the total polymer in the expandable sheath. In another non-limiting configuration, the expandable sheath includes a first polymer layer and a second polymer layer, wherein the first and second polymer layers are formed of different polymers. In another non-limiting configuration, the expandable sheath includes a first polymer layer and a second polymer layer, wherein the first and second polymer layers are formed of different polymers, and wherein the thickness of the layers is different (e.g., inner polymer layer thickness is 0.001-0.01 inches and all values and ranges therebetween, outer polymer layer thickness is 0.005-0.03 inches and all values and ranges therebetween). In another non-limiting configuration, the expandable sheath includes a first polymer layer and a second polymer layer, wherein the first and second polymer layers are formed of different polymers, and wherein the thickness of the inner polymer layer (e.g., first polymer layer) is less than a thickness of the outer polymer layer (e.g., second polymer layer).

In another and/or alternative non-limiting aspect of the present disclosure, the expandable sheath is partially or fully formed of a polymer material and wherein the expandable sheath includes at least two polymers, wherein a portion of the expandable sheath is fully or partially formed of a polymer that forms a generally rigid structure and another flexible portion of the expandable sheath that is connected to the generally rigid structure that is partially or fully formed from of a flexible or elastomeric polymer (e.g., natural rubbers, styrene-butadiene block copolymers, polyisoprene, polybutadiene, ethylene propylene rubber, ethylene propylene diene rubber, silicone elastomers, fluoroelastomers, polyurethane elastomers, nitrile rubbers, etc.), and wherein the durometer of the flexible or elastomeric polymer is less (e.g., 1.2-2000 times less and all values and ranges therebetween) than the durometer of the polymer used to partially or fully form the generally rigid structure. In another non-limiting embodiment, the expandable sheath that includes the generally rigid structure is configured to generally maintain its shape and size when the expandable sheath expands from a first outer diameter or first outer cross-sectional area to a second outer diameter or second outer cross-sectional area as a medical device is moved through an interior passageway in the expandable sheath, and to also generally maintain its shape and size when the expandable sheath partially or fully returns to the first outer diameter or first outer cross-sectional area once the medical device has passed partially or fully through the expandable sheath. In another non-limiting embodiment, the expandable sheath that includes the flexible portion is configured to change its shape and/or size when the expandable sheath expands from a first outer diameter or first outer cross-sectional area to a second outer diameter or second outer cross-sectional area as a medical device is moved through an interior passageway in the expandable sheath, and to also fully change back or closely change back to its original shape and/or size when the expandable sheath partially or fully returns to the first outer diameter or first outer cross-sectional area once the medical device has passed partially or fully through the expandable sheath. In another non-limiting embodiment, the expandable sheath includes one or more flexible portions and one or more generally rigid structure. In one non-limiting configuration, the expandable sheath that includes at least one flexible portions and two or generally rigid structures, and wherein one of the flexible portions is connected to two of the generally rigid structures. In another non-limiting embodiment, one or more of the flexible portions includes an additional shape memory material. Non-limiting shape member material includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X=Zr, B, Cr, Gd) alloy, l) Cu—Al—Ni—Hf alloy, m) Cu—Sn alloy, n) Cu—Zn alloy, o) Cu—Zn—X (X=Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly (ethyleneglycol) dimethacrylate (tBA-co-PEGDMA) polymers, etc.), z) (meth) acrylate-based SMPs, aa) polyurethane-based SMPs, and/or bb) blends of polyurethane and polyvinylchloride-based SMPs. As defined herein, the flexible or elastomeric polymer is not a shape memory material. In another non-limiting embodiment, when the shape memory material is included in one or more of the flexible portions, the shape memory material can include 5-100 wt. % (and all values and ranges therebetween) of each of the flexible portions that are formed of or include the shape memory material. When the flexible portion is not fully formed of the shape memory material, the flexible or elastomeric polymer can partially or fully coat the outer surface of the shape memory material. In another non-limiting embodiment, the generally rigid structure can optionally include a shape memory material. When the shape memory material is included in the one or more generally rigid structures, the shape memory material can include 1-60 wt. % (and all values and ranges therebetween) of each of the generally rigid structures that include the shape memory material.

In another and/or alternative non-limiting aspect of the present disclosure, the expandable sheath is partially formed of a shape memory material. In one non-limiting arrangement, the expandable sheath includes a shape memory polymer material. In another non-limiting arrangement, the expandable sheath includes a shape memory metal. In another non-limiting arrangement, the expandable sheath includes a shape memory metal and is coated with a polymer material. In one non-limiting configuration, the expandable sheath includes a shape memory metal, which shape memory material is or includes a nickel-titanium alloy. In another non-limiting configuration, the shape memory material is partially or fully encapsulated in a non-shape memory polymer layer, or the shape memory material is partially or fully encapsulated in two or more layers of a non-shape memory polymer layer, wherein the polymer layers can be formed of the same or different polymer material.

In another and/or alternative non-limiting aspect of the present disclosure, the expandable sheath has a generally circular cross-sectional outer profile and/or inner cavity profile when the expandable sheath is in a first outer diameter or first outer cross-sectional area and/or when the expandable sheath is expanded to a second outer diameter or second outer cross-sectional area as a medical device is moved through an interior passageway in the expandable sheath.

In another and/or alternative non-limiting aspect of the present disclosure, the expandable sheath can optionally include a hemostasis valve at or near one end of the expandable sheath.

In another and/or alternative non-limiting aspect of the present disclosure, the outer perimeter of the expandable sheath can optionally have a constant cross-sectional size and shape along 50-100% (and all values and ranges therebetween) of the longitudinal length of the expandable sheath when the expandable sheath is in the contracted orientation and/or when the expandable sheath is in the expanded orientation; and typically the outer perimeter expandable sheath has a constant cross-sectional size and shape along 75-99% of the longitudinal length of the expandable sheath when the expandable sheath is in the contracted orientation and/or when the expandable sheath is in the expanded orientation. In another non-limiting embodiment, the outer surface of the expandable sheath and/or the interior surface of the expandable sheath has a constant size and shape along 50-100% (and all values and ranges therebetween) of the longitudinal length of the expandable sheath when the expandable sheath is in the contracted orientation and/or when the expandable sheath is in the expanded orientation; and typically the outer surface of the expandable sheath and/or the interior surface of the expandable sheath has a constant size and shape along 75-99% of the longitudinal length of the expandable sheath when the expandable sheath is in the contracted orientation and/or when the expandable sheath is in the expanded orientation. In another non-limiting embodiment, the shape of the outer perimeter of the expandable sheath when in the fully expanded orientation is a circular cross-sectional shape.

In another and/or alternative non-limiting aspect of the present disclosure, the interior surface (i.e., the surface of the interior cavity of the expandable sheath) and/or exterior surface of the expandable sheath can optionally be coated with one or more polymers, drugs, etc.

In another and/or alternative non-limiting aspect of the present disclosure, the interior surface or cavity and/or the outer surface of the expandable sheath can optionally be coated with one or more polymers. In one non-limiting embodiment, the interior surface or cavity of the expandable sheath and/or the outer surface of the expandable sheath includes one or more polymers to reduce friction between a) the medical device and the interior cavity of the expandable sheath as the medical device move through the expandable sheath, and/or b) the outer surface the expandable sheath and the interior surface of the body passageway (e.g., blood vessel, heart, etc.) as the expandable sheath is moved within the body passageway.

In another and/or alternative non-limiting aspect of the present disclosure, the interior cavity and/or outer surface of the expandable sheath can optionally be coated with one or more polymers, drugs, etc. In one non-limiting embodiment, one or more polymers are coated on the outer surface of the expandable can include a hydrophilic coating to facilitate in the insertion of the expandable sheath into body passageway (e.g., blood vessel, heart, etc.).

In another and/or alternative non-limiting aspect of the present disclosure, the expandable sheath can be partially or fully formed by an extrusion process, stamping process and/or casting process. In one non-limiting embodiment, the expandable sheath is formed by an extrusion process.

In another and/or alternative non-limiting aspect of the present disclosure, the expandable sheath includes multiple layers. In non-limiting embodiment, the expandable sheath includes an outer layer portion, a frame, and an inner layer portion. In one non-limiting arrangement the outer layer portion, the frame, and the inner layer portion facilitate in maintaining the shape and integrity of the expandable sheath. In one non-limiting embodiment, the frame is partially or fully formed of a shape memory material.

In another and/or alternative non-limiting aspect of the present disclosure, the expandable sheath includes an inner layer portion that can be formed of one or more polymer layers. In one non-limiting embodiment, the one or more polymer layers used to partially or fully form the inner layer portion can include polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks, or combinations thereof. In one non-limiting configuration, the inner layer portion is or includes polytetrafluoroethylene. In another non-limiting embodiment, the inner layer portion is formed of a single polymer layer. In another non-limiting embodiment, the inner layer portion is formed of at least two polymer layers. When the inner layer portion includes two or more layers, the composition of the layers can be the same or different. In another non-limiting embodiment, the thickness of the inner layer portion is generally at least 0.001 inches, typically 0.001-0.02 inches (and all values and ranges therebetween), and more typically 0.0015-0.005 inches. In another non-limiting embodiment, the inner layer portion is formed of a material that allows the outer diameter or outer cross-sectional area of the expandable sheath and the diameter or cross-sectional area of the interior passageway to expand and contract as the medical device is moved through the expandable sheath without damaging the material that forms inner layer portion. In another non-limiting embodiment, when the inner layer portion is formed of two or more layers, the inner layer portion can optionally be formed by a co-extrusion process.

In another and/or alternative non-limiting aspect of the present disclosure, the expandable sheath includes an outer layer portion that can be formed of one or more polymer layers. The outer layer portion can be formed of the same or different material from the inner layer portion. In one non-limiting embodiment, the one or more polymer layers used to partially or fully form the outer layer portion can include polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks, or combinations thereof. In one non-limiting configuration, the outer layer portion is or includes block copolymers made up of polyamide blocks and polyether blocks. In another non-limiting embodiment, the outer layer portion is formed of a single polymer layer. In another non-limiting embodiment, the outer layer portion is formed of at least two polymer layers. When the outer layer portion includes two or more layers, the composition of the layers can be the same or different. In another non-limiting embodiment, the thickness of the outer layer portion is generally at least 0.002 inches, typically 0.002-0.03 inches (and all values and ranges therebetween), and more typically 0.004-0.02 inches. In another non-limiting embodiment, the thickness of the outer layer portion is greater than the thickness of the inner layer portion. In one non-limiting arrangement, the thickness of the outer layer portion is 2-10 times greater (and all values and ranges therebetween) than the thickness of the inner layer portion. In one non-limiting arrangement, the thickness of the outer layer portion is 4-8 times greater than the thickness of the inner layer portion. In another non-limiting embodiment, the outer layer portion is formed of a material that allows the outer diameter or outer cross-sectional area of the expandable sheath and the diameter or cross-sectional area of the interior passageway to expand and contract as the medical device is moved through the expandable sheath without damaging the material that forms the outer layer portion. In another non-limiting embodiment, when the outer layer portion is formed of two or more layers, the outer layer portion can optionally be formed by a co-extrusion process. In another non-limiting embodiment, the outer layer portion is formed of a different polymer from the polymer used to form the inner layer portion.

In another and/or alternative non-limiting aspect of the present disclosure, the expandable sheath optionally includes a frame. The thickness of the frame of the expandable sheath is generally at least 0.002 inches, typically 0.002-0.03 inches (and all values and ranges therebetween), and more typically 0.004-0.012 inches. In another non-limiting embodiment, the thickness of the frame is greater than the thickness of the inner layer portion (e.g., polymer layer). In another non-limiting embodiment, the thickness of the frame is equal to or less than the thickness of the outer layer portion. In one non-limiting configuration, the thickness of the frame is less than the thickness of the outer layer portion (e.g., polymer layer). In one non-limiting embodiment, the frame is partially or fully formed of a shape memory material (e.g., Nitnol™, etc.).

In another and/or alternative non-limiting aspect of the present disclosure, the expandable sheath optionally includes a frame. The frame of the expandable sheath has a configuration that allows the outer diameter or outer cross-sectional area of the expandable sheath and the diameter or cross-sectional area of the interior passageway to expand and contract as the medical device is moved through the expandable sheath without damaging the frame. In one non-limiting embodiment, the frame provides strength, structure, and/or shape to the expandable sheath. The frame can be formed of one or more pieces of material. In one embodiment, the frame is a single structure that extends 5-100% (and all values and ranges therebetween) the longitudinal length of the expandable sheath. The single structure can be formed of one piece of material, or a plurality of materials connected together. In another non-limiting embodiment, the frame is multiple structures that are spaced form one another along a longitudinal length of the expandable sheath. The sum of the multiple structures that form the frame extend 5-100% (and all values and ranges therebetween) the longitudinal length of the expandable sheath. The size, shape and/or material of each of the multiple structures that form the frame can be the same or different. In another non-limiting embodiment, the material that is used to partially or fully form the frame is a plastic material, a metal material, a composite material, a fiber composite material, etc. In another non-limiting embodiment, the material that is used to partially or fully form the frame is or includes a shape memory material.

In another and/or alternative non-limiting aspect of the present disclosure, the expandable sheath optionally includes a frame wherein the frame is formed of a single wire that has been bent to form multiple symmetrically oriented arm sections along the longitudinal length of the frame, and wherein a majority (e.g., 50.0001-100% and all values and ranges therebetween) or all of the wire that forms the frame does not overlap along the transverse axis (i.e. the axis that is transverse to the longitudinal axis of the frame) when the frame is in the expanded and/or contracted positions. In another non-limiting embodiment, the frame is formed of a single wire that has been bent to form multiple symmetrically oriented arm sections along the longitudinal length of the frame, and wherein a majority (e.g., 50.0001-100% and all values and ranges therebetween) or all of the arm sections of the frame overlap one another along the longitudinal axis of the frame when the frame is in the expanded and/or contracted positions. In one non-limiting configuration, the frame forms a plurality of crescent shapes along the longitudinal length of the frame when in the expanded position. In another non-limiting configuration, for a plurality of the crescent shapes, A) each side of the crescent shape is formed of two wire portions that are bent in a U-shape for the crescent tip portion and wherein i) the U-shape of the crescent tip portion has a radius of curvature that is 1.5-30 times (and all values and ranges therebetween) less than a radius of curvature of the base portion of the arm section when the frame is in the expanded position, and ii) the crescent tip portion lies in a plane that is ±10º (and all values and ranges therebetween) to the longitudinal axis of the frame, and B) the base portion of the arm section lies in a plane that is 65-88° (and all values and ranges therebetween) to the longitudinal axis of the frame. In another non-limiting embodiment, wherein a majority (e.g., 50.0001-100% and all values and ranges therebetween) or all of the tip portions of the frame overlap one another along the longitudinal axis of the frame when the frame is in the contracted position, and wherein a majority (e.g., 50.0001-100% and all values and ranges therebetween) or all of the tip portions of the frame do not overlap one another when the frame is in the expanded position. In another non-limiting embodiment, the pattern and shape of the frame enables the outer diameter or outer cross-sectional area of the expandable sheath and the diameter or cross-sectional area of the interior passageway to expand and contract as the medical device is moved through the expandable sheath without damaging the frame. In another non-limiting embodiment, the shape memory material used to at least partially form the frame causes the expandable sheath to contract to its original or near original shape after being expanded as the medical device passes through the expandable sheath. In another non-limiting embodiment, the wire of the frame has a generally constant size and cross-sectional area and shape, and the frame is generally formed of a single piece of wire; however, this is not required.

In another and/or alternative non-limiting aspect of the present disclosure, the expandable sheath optionally includes a frame wherein the frame is partially or fully formed of a sheet of material or a wire that includes one or more folded portions or curved portions about the perimeter of the expandable sheath. The one or more curved or folded portions are configured to partially or fully unfold and/or partially or fully straighten so as to increase in length about the perimeter of the expandable sheath when the expandable sheath is expanded. The one or more curved or folded portions can be formed of or include a shape memory material that facilitates in the one or more curved or folded portions reshaping into a folded or curved configuration when the expandable sheath become unexpanded.

In another and/or alternative non-limiting aspect of the present disclosure, the expandable sheath optionally includes a frame wherein the frame is partially or fully formed of a sheet of material or a wire that includes a plurality of folded or curved sections that are spaced from one another about the perimeter of the expandable sheath. The one or more curved or folded portions are configured to partially or fully unfold and/or partially or fully straighten so as to increase in length about the perimeter of the expandable sheath when the expandable sheath is expanded. The one or more curved or folded portions can be formed of or include a shape memory material that facilitates in the one or more curved or folded portions reshaping into a folded or curved configuration when the expandable sheath become unexpanded.

In another and/or alternative non-limiting aspect of the present disclosure, one or both ends of the expandable sheath are optionally subjected to a connection processed to connect together the outer layer portion and the inner layer portion. One non-limiting connection process is a reflow process to secure the outer layer portion and the inner layer portion by a heat bonding process (e.g., heat fusion process or heat melt process). As can be appreciated, additional or alternative connection processes can be used (e.g., adhesive, mechanical connection, crimping, etc.). When the outer and inner layer portions are assembled together, the frame can be partially or fully positioned between the inner and outer layer portions. When the inner and outer layer portions are secured together (e.g., reflowed, adhesive, melted seam, pressure connection, etc.) at one or both ends of the expandable sheath, the frame is also caused to be fixed in position between the inner and outer layer portions.

In another and/or alternative non-limiting aspect of the present disclosure, the expandable sheath is optionally processed to connect together the outer layer portion and the inner layer portion. In one non-limiting embodiment, 51-100% (and all values and ranges therebetween) of the longitudinal length sheath is subjected to the connection process. In another non-limiting embodiment, the connection process includes a heat or reflow process. In another non-limiting embodiment, the frame is maintained in the full or partial expanded positioned during the connection process.

In another and/or alternative non-limiting aspect of the present disclosure, the inner surface of the inner layer portion of the expandable sheath can optionally be or include a lubrication coating and/or liner to facilitate in the movement of a medical device through the expandable sheath. In one non-limiting embodiment, the inner surface of the inner layer portion has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the inner surface of the inner layer portion includes a coating of PTFE.

In another and/or alternative non-limiting aspect of the present disclosure, the outer surface of the outer layer portion of the expandable sheath can optionally be or include a lubrication coating and/or liner to facilitate in the movement of the expandable sheath in a body passageway. In one non-limiting embodiment, the outer surface of the outer layer portion has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the outer surface of the outer layer portion includes a coating of PTFE.

In another and/or alternative non-limiting aspect of the present disclosure, the outer surface of the outer layer portion of the expandable sheath can optionally include a hydrophilic coating to facilitate in the insertion of the expandable sheath into body passageway. Non-limiting examples of hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands).

In another and/or alternative non-limiting aspect of the present disclosure, the expandable sheath optionally includes one or more radiopaque marker or fillers. The radiopaque filler or marker (when used) can be located on the outer surface of the expandable sheath, embedded on one or more layers of the expandable sheath, and/or be located between on or more layers of the expandable sheath. Non-limiting materials that can be used as a radiopaque filler or marker include barium sulfite, bismuth trioxide, titanium dioxide, and/or bismuth subcarbonate.

In another and/or alternative non-limiting aspect of the present disclosure, the inner diameter of the cavity through the longitudinal length of the expandable sheath prior to the expandable sheath being expanded is 0.08-0.2 inches (and all values and ranges therebetween), and the maximum inner diameter of the cavity through the longitudinal length of the expandable sheath after the expandable sheath is expanded is at least 0.15-0.3 inches (and all values and ranges therebetween).

In another and/or alternative non-limiting aspect of the present disclosure, the wall thickness of the expandable sheath is generally less than 0.8 mm, and typically less than 0.5 mm.

In another and/or alternative non-limiting aspect of the present disclosure, the longitudinal length of the expandable sheath is at least 5 inches and typically 5-40 inches (and all values and ranges therebetween).

In another and/or alternative non-limiting aspect of the present disclosure, the shape of the expandable sheath can optionally be tubular-shaped or a cylindrical tube. The shape and size of the expandable sheath can be uniform along a majority (e.g., 60-99.99% and all values and ranges therebetween) or the full longitudinal length of the expandable sheath.

In another and/or alternative non-limiting aspect of the present disclosure, the expandable sheath in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through sheath, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

In another and/or alternative non-limiting aspect of the present disclosure, the expandable sheath in accordance with the present disclosure optionally includes a conical-shaped front portion that tapers in size from the front end of the expandable sheath to the generally tubular-shaped portion of the expandable sheath. The conical-shaped front portion includes an opening therethrough such that a medical device can be inserted into the conical-shaped front portion and into the cavity of the generally tubular-shaped portion of the expandable sheath. In another non-limiting embodiment, 0-50% (and all values and ranges therebetween) of the longitudinal length of the conical-shaped front portion is absent the frame of the expandable sheath. The maximum cross-sectional area of the cavity of the conical-shaped front portion is generally at least 1.5 times (e.g., 1.5-10 times and all values and ranges therebetween) the cross-sectional area of the internal passageway or cavity of the tubular portion of the expandable sheath when in the collapsed or unexpanded position. In another non-limiting embodiment, the conical-shaped front portion can be formed of the same or different material as the polymer used to form the inner and outer layers of the tubular portion of the expandable sheath.

In another and/or alternative non-limiting aspect of the present disclosure, the expandable sheath includes an optional lip that overlaps a portion of the outer surface of the tubular body of the expandable sheath when the expandable sheath is in the collapsed or unexpanded position. All or a portion of the lip can optionally have a thickness that is less than a thickness of the outer layer of the expandable sheath. The lip can be formed of the same or different material than the outer layer of the expandable sheath. The lip has a length that is 2-50% (and all values and ranges there therebetween) the length of the outer perimeter of the tubular body of the expandable sheath. In one non-limiting embodiment, the lip overlaps a portion of the outer surface of the body of the expandable sheath when the expandable sheath is in the collapsed or unexpanded position. In another non-limiting embodiment, the lip does not overlaps a portion of the outer surface of the body of the expandable sheath when the expandable sheath is in the fully expanded position.

In another and/or alternative non-limiting aspect of the present disclosure, the expandable sheath includes one or more breakable connection points or regions on the outer layer of the expandable sheath and/or on the outer layer of the expandable sheath and the optional lip to maintain the expandable sheath in the collapsed or unexpanded position until a medical device is inserted through the internal passageway or cavity of the tubular portion of the expandable sheath to cause the expandable sheath to move to the non-collapsed or expanded position. The one or more breakable connection points or regions can be formed of a) an adhesive, b) one or more melted portions that optionally have a thickness that is less than a thickness of the wall of the expandable sheath (e.g., 1-25% a thickness of the wall of the expandable sheath and all values and ranges therebetween), c) breakable stitching, d) hook and look fastener, and/or e) mechanical and/or friction connection. When a plurality of breakable connection points are located on the expandable sheath, the plurality of breakable connection points are generally spaced from one another. As can be appreciated, one or more of the breakable connection points or regions can extend 5-100% (and all values and ranges therebetween) of the longitudinal length of the expandable sheath.

In another and/or alternative non-limiting aspect of the present disclosure, the expandable sheath is formed by 1) providing a forming rod that has a diameter that is ±40% (and all values and ranges therebetween) the cavity diameter or cross-sectional area of the cavity or passageway when the tubular portion of the expandable sheath is in the fully expanded position; 2) positioning an inner polymer layer about the outer surface of the forming rod; 3) positioning the frame about the inner polymer layer; 4) positioning the outer polymer layer about the frame; 5) partially or fully connecting together the inner and outer polymer layers; and 6) removing the forming rod from the inner layer of the formed expandable sheath. As defined herein, the expandable sheath is in the fully expanded position when the cavity diameter or cross-sectional area of the cavity or passageway of the tubular member has increased 1.5-6 times (and all values and ranges therebetween) to the cavity diameter or cross-sectional area of the cavity or passageway when the tubular portion of the expandable sheath in the collapsed or unexpanded position. One or more optional steps can be used to form the expandable sheath such as, but not limited to, a) cutting a portion or otherwise forming an opening in the inner layer along the longitudinal length of the tubular portion of the expandable sheath that partially or fully aligns with the with the opening of the frame when the frame is positioned about the forming rod, b) cutting a portion or otherwise forming an opening in the outer layer along the longitudinal length of the tubular portion of the expandable sheath that partially or fully aligns with the with the opening of the frame when the frame is positioned about the forming rod, c) forming one or more breakable connections on the outer layer to facilitate in maintaining the expandable sheath in the unexpanded position, d) forming a tab on the outer layer that is configured to overlap a portion of the outer layer when the expandable sheath is in the unexpanded position, e) heating or reflowing the inner and outer layers along a portion or all of the longitudinal length of the tubular portion of the expandable sheath to connect together the inner and outer layers, f) connecting a conical-shaped portion to a front end of the tubular member of the expandable sheath, and/or g) applying a layer of material over a portion of the frame that is located between the gap or opening in the outer layer, so that during the heating or flow process, portions of the outer layer to not flow on to the exposed frame.

One non-limiting object of the disclosure, there is the provision of an expandable sheath.

Another and/or alternative non-limiting object of the disclosure is the provision of an expandable sheath that is usable with a medical device and which expandable sheath is expandable in at least the regions where the medical device is positioned in the expandable sheath and which sheath reforms to its same or similar size and shape after the medical device has passed through a portion or all of the expandable sheath.

Another and/or alternative non-limiting object of the disclosure is the provision of an expandable sheath that can be used to protect the body passageway of a patient as a medical device is inserted into and/or through the body passageway and to a treatment site.

Another and/or alternative non-limiting object of the disclosure is the provision of an expandable sheath that can be used with catheter arrangements for repairing and/or replacing heart valves.

Another and/or alternative non-limiting object of the disclosure is the provision of an expandable sheath that can be used to reduce or minimize trauma to a body passageway (e.g., blood vessel, duct, etc.) by allowing for temporary expansion of a portion of the expandable sheath to accommodate at least a portion of the delivery system and/or medical device, followed by a return to the same or similar original diameter once the delivery system and/or medical device has partially or fully passes through the expandable sheath.

Another and/or alternative non-limiting object of the disclosure is the provision of an expandable sheath that can be configured to progressively increase in outer diameter and then progressively decrease in outer diameter as the medical device moves through the interior passageway of the expandable sheath.

Another and/or alternative non-limiting object of the disclosure is the provision of an expandable sheath that can be configured such that the outer diameter or cross-sectional area of the expandable sheath and the diameter or cross-sectional area of the interior passageway expands to a second diameter along the longitudinal length of the expandable sheath as the medical device moves through the expandable sheath, and then partially or fully returns to the first outer diameter once the medical device has fully passes through the expandable sheath.

Another and/or alternative non-limiting object of the disclosure is the provision of an expandable sheath that is partially for fully formed of a shape memory material to facilitate in causing the expandable sheath to partially or fully return to the first outer diameter once the medical device has partially or fully passed through the expandable sheath.

Another and/or alternative non-limiting object of the disclosure is the provision of an expandable sheath that provides access to a body passageway for delivering a medical device into the body passageway; the expandable sheath is expandable from a first diameter to a second diameter; the second diameter is greater than the first diameter; the expandable sheath including an internal cavity that extends a longitudinal length of the expandable sheath; the expandable sheath includes a shape memory frame that is partially or fully encapsulated in one or more polymer layers.

Another and/or alternative non-limiting object of the disclosure is the provision of an expandable sheath that is configured to expand to the second diameter during movement of the medical device through the internal cavity of the expandable sheath; the expandable device is configured to contract from the second diameter to the first diameter after the medial device partially or fully passes through the internal cavity of the expandable sheath.

Another and/or alternative non-limiting object of the disclosure is the provision of an expandable sheath that includes a single frame and wherein the frame is formed of a single piece of shape memory material.

Another and/or alternative non-limiting object of the disclosure is the provision of an expandable sheath wherein the polymer material includes one or more polymers selected from the group consisting of polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks.

Another and/or alternative non-limiting object of the disclosure is the provision of an expandable sheath wherein the frame includes one or more material selected from the group consisting of a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X=Zr, B, Cr, Gd) alloy, l) Cu—Al—Ni—Hf alloy, m) Cu—Sn alloy, n) Cu—Zn alloy, o) Cu—Zn—X (X=Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, and x) Ti—Nb alloy.

Another and/or alternative non-limiting object of the disclosure is the provision of an expandable sheath wherein the expandable sheath includes two polymer layers, and wherein the first polymer layer constitutes 5-49 wt. % of the total polymer in the expandable sheath, and the second polymer layer constitutes 51-95 wt. % of the total polymer in the expandable sheath.

Another and/or alternative non-limiting object of the disclosure is the provision of an expandable sheath wherein the expandable sheath in the fully contracted orientation has a cross-sectional wall thickness that is generally the same for 70-98% of a circumference of the expandable sheath.

Another and/or alternative non-limiting object of the disclosure is the provision of an expandable sheath wherein the expandable sheath includes one or more radiopaque fillers or markers.

Another and/or alternative non-limiting object of the disclosure is the provision of an expandable sheath wherein the expandable sheath is at least partially or fully formed by an extrusion process, stamping process, reforming or heat process, 3D printer process, and/or casting process.

Another and/or alternative non-limiting object of the disclosure is the provision of a method of introducing a prosthetic heart valve, stent, catheter, and/or other type of medical device into a patient's vasculature, the method comprising: a) optionally forming an incision in a patient; b) inserting an expandable sheath through the optional incision and at least partially into a blood vessel of a patient; the expandable sheath expandable from a first diameter to a second diameter; the second diameter greater than the first diameter; the expandable sheath including an internal cavity that extends a longitudinal length of the expandable sheath; the expandable sheath includes one or more polymer layers and a frame that is at least partially or fully encapsulated in the one or more polymer layers; and c) advancing the prosthetic heart valve, stent, catheter, and/or other type of medical device through the internal cavity of the expandable sheath to cause the expandable sheath to expand from the first diameter to the second diameter as the prosthetic heart valve, stent, catheter, and/or other type of medical device partially or fully passes through the internal cavity of the expandable sheath.

Another and/or alternative non-limiting object of the disclosure is the provision of method of introducing a prosthetic heart valve into and through the expandable sheath, and into a patient's vasculature, and wherein the prosthetic heart valve is a stent-mounted heart valve mounted in a radially crimped state on a delivery apparatus.

Another and/or alternative non-limiting object of the disclosure is the provision of method of introducing a prosthetic heart valve, stent, catheter, and/or other type of medical device into a patient's vasculature, and wherein the expandable sheath contracts from the second diameter to the first diameter after the prosthetic heart valve, stent, catheter, and/or other type of medical device has partially or fully passed through the internal cavity of the expandable sheath.

Another and/or alternative non-limiting object of the disclosure is the provision that the frame is formed of a single wire material.

Another and/or alternative non-limiting object of the disclosure is the provision that the frame is formed of a shape memory material.

Another and/or alternative non-limiting object of the disclosure is the provision that the polymer material constitutes 40-100% of the expandable sheath.

Another and/or alternative non-limiting object of the disclosure is the provision that the expandable sheath has a constant cross-sectional size and/or shape along 50-100% of a longitudinal length of the expandable sheath when the expandable sheath is in the fully contracted orientation and/or when the expandable sheath is in the fully expanded orientation.

Another and/or alternative non-limiting object of the disclosure is the provision that an outer cross-sectional profile of the expandable sheath in the fully expanded orientation and/or the fully contracted orientation is generally circular.

Another and/or alternative non-limiting object of the disclosure is the provision that the cross-sectional shape of an internal cavity of the expandable sheath in the fully contracted orientation is generally circular.

Another and/or alternative non-limiting object of the disclosure is the provision of a method of introducing a prosthetic heart valve, stent, catheter, and/or other type of medical device into a patient's vasculature, the method comprising: a) inserting an expandable sheath at least partially into a blood vessel of a patient; and b) advancing the prosthetic heart valve, stent, catheter, and/or other type of medical device through a central lumen of the expandable sheath to cause the expandable sheath to expand from an outwardly directed radial force of the prosthetic heart valve, stent, catheter, and/or other type of medical device exerted against the expandable sheath, and then the expandable sheath at least partially contracts as the prosthetic heart valve, stent, catheter, and/or other type of medical device passes through the expandable sheath.

Another and/or alternative non-limiting object of the disclosure is the provision of an expandable sheath that provides access to the vasculature for delivering of medical devices or for medical intervention, the expandable sheath includes: a) an inner polymer layer, b) an outer polymer layer, and c) a frame that is partially or fully encapsulated by the inner and/or outer polymer layers; wherein the expandable sheath is expandable between a fully contracted orientation and a fully expanded orientation; and wherein the expandable sheath opens for the delivery or removal of medical devices or for medical intervention and closes and/or retracts to a smaller diameter once medical devices distally pass or are proximally removed from the expandable sheath.

Another and/or alternative non-limiting object of the disclosure is the provision of an expandable sheath that is adapted for use in the delivery of a medical device into a body passageway; the sheath comprising a) an outer layer portion; b) an inner layer portion; and c) a frame; the frame at least partially positioned between the inner and outer layer portions; and wherein the sheath is configured to expand from a first outer diameter to a second outer diameter as the medical device is moved through the sheath, and thereafter the sheath partially or fully returns to the first outer diameter once the medical device has passed partially or fully through the sheath.

Another and/or alternative non-limiting object of the disclosure is the provision of an expandable sheath that includes an internal cavity extending the longitudinal length of the sheath.

Another and/or alternative non-limiting object of the disclosure is the provision an expandable sheath that has a cylindrical shape.

Another and/or alternative non-limiting object of the disclosure is the provision of an expandable sheath wherein the outer layer portion includes a polymer material.

Another and/or alternative non-limiting object of the disclosure is the provision of an expandable sheath wherein the outer layer portion includes a first and second polymer layer.

Another and/or alternative non-limiting object of the disclosure is the provision of an expandable sheath wherein the first and second polymer layers of the outer layer are formed of a different polymer.

Another and/or alternative non-limiting object of the disclosure is the provision of an expandable sheath wherein a thickness of the outer layer portion is greater than a thickness of the inner layer portion.

Another and/or alternative non-limiting object of the disclosure is the provision of an expandable sheath wherein an outer surface of the outer layer portion includes a friction-reducing coating or layer; the friction-reducing coating or layer optionally has a coefficient of friction of no more than 0.1.

Another and/or alternative non-limiting object of the disclosure is the provision of an expandable sheath wherein the inner layer portion includes a polymer material.

Another and/or alternative non-limiting object of the disclosure is the provision an expandable sheath wherein an inner surface of the inner layer portion includes a friction-reducing coating or layer; the friction-reducing coating or layer has a coefficient of friction of optionally no more than 0.1.

Another and/or alternative non-limiting object of the disclosure is the provision of an expandable sheath wherein the frame includes a shape memory material.

Another and/or alternative non-limiting object of the disclosure is the provision of an expandable sheath wherein the shape memory material includes a nickel-titanium alloy.

Another and/or alternative non-limiting object of the disclosure is the provision of an expandable sheath wherein the frame is at least partially positioned between the inner and outer layer portions.

Another and/or alternative non-limiting object of the disclosure is the provision of an expandable sheath wherein the inner and outer layer portions are subjected to a reflow process to connect together the inner and outer layer portions.

Another and/or alternative non-limiting object of the disclosure is the provision of an expandable sheath that optionally includes a conical-shaped front portion that tapers in size from the front end of the expandable sheath to the generally tubular-shaped portion of the expandable sheath. The conical-shaped front portion includes an opening therethrough to facilitate in the insertion of the medical device into the conical-shaped front portion and into the cavity of the generally tubular-shaped portion of the expandable sheath.

Another and/or alternative non-limiting object of the disclosure is the provision of an expandable sheath that includes an optional lip that overlaps a portion of the outer surface of the tubular body of the expandable sheath when the expandable sheath is in the collapsed or unexpanded position, and optionally does not overlap a portion of the outer surface of the body of the expandable sheath when the expandable sheath is in the fully expanded position.

Another and/or alternative non-limiting object of the disclosure is the provision of an expandable sheath that optionally includes one or more breakable connection points. The one or more optional breakable connection points can be located a) on the outer layer of the expandable sheath and/or b) on the outer layer of the expandable sheath and the optional lip. The one or more optional breakable connection points can be used to maintain the expandable sheath in the collapsed or unexpanded position until a medical device is inserted through the internal passageway or cavity of the tubular-shaped portion of the expandable sheath to cause the expandable sheath to move to the non-collapsed or expanded position.

Another and/or alternative non-limiting object of the disclosure is the provision of a method for forming an expandable sheath by 1) providing a forming rod; 2) positioning an inner polymer layer about the outer surface of the forming rod; 3) positioning a frame about the inner polymer layer; 4) positioning an outer polymer layer about the frame; 5) partially or fully connecting together the inner and outer polymer layers; and 6) removing the forming rod from the inner layer of the formed expandable sheath.

Another and/or alternative non-limiting object of the disclosure is the provision of a method for forming an expandable sheath that optionally includes one or more steps of a) cutting a portion or otherwise forming an opening in the inner layer along the longitudinal length of the tubular-shaped portion of the expandable sheath that partially or fully aligns with the with the opening of the frame when the frame is positioned about the forming rod, b) cutting a portion or otherwise forming an opening in the outer layer along the longitudinal length of the tubular-shaped portion of the expandable sheath that partially or fully aligns with the with the opening of the frame when the frame is positioned about the forming rod, c) forming one or more breakable connections on the outer layer to facilitate in maintaining the expandable sheath in the unexpanded position, d) forming a tab on the outer layer that is configured to overlap a portion of the outer layer when the expandable sheath is in the unexpanded position, e) heating or reflowing the inner and outer layers along a portion or all of the longitudinal length of the tubular portion of the expandable sheath to connect together the inner and outer layers, f) connecting a conical-shaped portion to a front end of the tubular-shaped member of the expandable sheath, and/or g) applying a layer of material over a portion of the frame that is located between the gap or opening in the outer layer so that during the heating or flow process, portions of the outer layer do not flow on to the exposed frame.

Another and/or alternative non-limiting object of the disclosure is the provision of a method of introducing a medical device into a body passageway of a patient wherein the step of inserting includes passing the expandable sheath transcutaneously through a surgically-created opening in the patient's skin such that at least a portion of the outer payer portion of the sheath is positioned adjacent to the surgically-created opening.

Another and/or alternative non-limiting object of the disclosure is the provision of a method of introducing a medical device into a body passageway of a patient further including the step of implanting the medical device at a treatment site within the patient.

Another and/or alternative non-limiting object of the disclosure is the provision of a method of introducing a medical device into a body passageway of a patient further including the step of expanding the medical device at the treatment site of the patient.

Another and/or alternative non-limiting object of the disclosure is the provision of an expandable sheath that is partially or fully formed of a polymer material and wherein the expandable sheath includes at least two polymers, wherein a portion of the expandable sheath is fully or partially formed of a polymer that forms a generally rigid structure and another flexible portion of the expandable sheath that is connected to the generally rigid structure that is partially or fully formed from of a flexible or elastomeric polymer, and wherein the durometer of the flexible or elastomeric polymer is less than the durometer of the polymer used to partially or fully form the generally rigid structure.

Another and/or alternative non-limiting object of the disclosure is the provision of an expandable sheath that includes one or more generally rigid structures that are configured to generally maintain their shape and size when the expandable sheath expands from a first outer diameter or first outer cross-sectional area to a second outer diameter or second outer cross-sectional area, and to also generally maintain its shape and size when the expandable sheath partially or fully returns to the first outer diameter or first outer cross-sectional area.

Another and/or alternative non-limiting object of the disclosure is the provision of an expandable sheath includes one or more flexible portions that are configured to change their shape and/or size when the expandable sheath expands from a first outer diameter or first outer cross-sectional area to a second outer diameter or second outer cross-sectional area, and to also fully change back or closely change back to their original shape and/or size when the expandable sheath partially or fully returns to the first outer diameter or first outer cross-sectional area.

Another and/or alternative non-limiting object of the disclosure is the provision of an expandable sheath includes one or more flexible portions and one or more generally rigid structure, and wherein one of the flexible portions is connected between to two of the generally rigid structures.

Another and/or alternative non-limiting object of the disclosure is the provision of an expandable sheath that includes one or more flexible portions that includes or is formed of a shape memory material.

Another and/or alternative non-limiting object of the disclosure is the provision of an expandable sheath that includes a frame, and wherein the frame is formed of one or more pieces of material.

Another and/or alternative non-limiting object of the disclosure is the provision of an expandable sheath that includes a frame, and wherein the frame is partially or fully formed of a sheet of material or a wire that includes one or more folded portions or curved portions about the perimeter of the expandable sheath, and wherein the one or more curved or folded portions are configured to partially or fully unfold and/or partially or fully straighten so as to increase in length about the perimeter of the expandable sheath when the expandable sheath is expanded.

Another and/or alternative non-limiting object of the disclosure is the provision of an expandable sheath that includes a frame, and wherein the frame is partially or fully formed of a sheet of material or a wire that includes one or more folded portions or curved portions about the perimeter of the expandable sheath, and wherein the one or more curved or folded portions can be formed of or include a shape memory material that facilitates in the one or more curved or folded portions reshaping into a folded or curved configuration when the expandable sheath become unexpanded.

Another and/or alternative non-limiting object of the disclosure is the provision of an expandable sheath that includes a frame, and wherein the frame is partially or fully formed of a sheet of material or a wire that includes one or more folded portions or curved portions about the perimeter of the expandable sheath, and wherein the one or more curved or folded portions are spaced from one another about the perimeter of the expandable sheath.

Other aspects, advantages and novel features of the present disclosure will become apparent from the following detailed description and figures of the disclosure.

### BRIEF DESCRIPTION OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Reference may now be made to the drawings, which illustrate various embodiments that the disclosure may take in physical form and in certain parts and arrangement of parts wherein:

FIG. 1 illustrates an expandable sheath in accordance with the present disclosure that can be used with a delivery apparatus for facilitating in delivering a medical device into a patient.

FIG. 2 is a side view of a portion of an expandable sheath in accordance with the present disclosure;

FIG. 3 is cross-sectional view of the expandable sheath illustrated in FIG. 2;

FIG. 4 is an isometric view of one non-limiting expandable sheath in accordance with the present disclosure in the unexpanded position;

FIG. 5 is an isometric view of the expandable sheath of FIG. 4 in the expanded position;

FIG. 6 is an isometric view of a non-limiting expandable frame in the unexpanded position that can be used in the expandable sheath in accordance with the present disclosure;

FIG. 7 is an isometric view of the expandable frame of FIG. 6 in the expanded position;

FIG. 8 is an end view of the expandable sheath of FIG. 4 in an unexpanded position;

FIG. 9 is an end view of a modified expandable sheath of FIG. 4 that includes an optional lip portion;

FIG. 10 is an end view of the expandable sheath of FIG. 8 in the expanded position;

FIG. 11 is an end view of the expandable sheath of FIG. 9 in the expanded position;

FIG. 12 is an end view of a modified expandable sheath of FIG. 8 that includes an optional expandable outer cover;

FIG. 13 is an end view of a modified expandable sheath of FIG. 10 that includes an optional expandable outer cover;

FIG. 14 is an end view of a modified expandable sheath of FIG. 8 that includes optional anti-kinking wires positioned along a portion or all of a longitudinal length of the expandable sheath and optional adhesion region used to facilitate in maintaining the expandable sheath in the unexpanded position;

FIG. 15 is an end view of a modified expandable sheath of FIG. 9 that includes optional anti-kinking wires positioned along a portion or all of a longitudinal length of the expandable sheath and optional adhesion region used to facilitate in maintaining the expandable sheath in the unexpanded position;

FIG. 16 is an end view of a modified expandable sheath of FIG. 12 that includes optional anti-kinking wires positioned along a portion or all of a longitudinal length of the expandable sheath and optional adhesion region used to facilitate in maintaining the expandable sheath in the unexpanded position;

FIG. 17 is a side view of an inner layer portion of the expandable sheath that is positioned about at least a portion of the outer surface of mandrel or forming rod;

FIG. 18 is a side view of an optional layer of plastic tape of the expandable sheath that can be used to inhibit or prevent the outer layer portion of the expandable sheath to flow into regions between the frame when the inner layer and outer layer are heated and connected together;

FIG. 19 is a side view of the outer layer of the expandable sheath that has been secured to the frame;

FIG. 20 is a side view of a portion of the expandable sheath that includes optional tack points, adhesion points or the like that facilitate in maintaining the expandable sheath in the unexpanded position;

FIG. 21 is a cross-sectional view of another non-limiting embodiment of an expandable sheath in the fully expanded position that is formed of multiple components that are connected together and optionally multiple materials;

FIG. 22 is a cross-sectional view of the expandable sheath of FIG. 21 in the partially expanded position;

FIG. 23 is a cross-sectional view of the expandable sheath of FIG. 21 in the unexpanded position;

FIG. 24 is a cross-sectional view of another non-limiting embodiment of an expandable sheath in the fully expanded position that is formed of a single piece of material;

FIG. 25 is a cross-sectional view of the expandable sheath of FIG. 21 and further includes an optional expandable outer layer;

FIG. 26 is a cross-sectional view of the expandable sheath of FIG. 22 and further includes an optional expandable outer layer;

FIG. 27 is a cross-sectional view of the expandable sheath of FIG. 23 and further includes an optional expandable outer layer;

FIG. 28 is a cross-sectional view of a portion of the expandable sheath of FIGS. 21-27 that illustrates that one or more portions can be formed of a core material that includes a coating;

FIG. 29 is a cross-sectional view of the expandable sheath of FIG. 23 and further optionally includes anti-kinking wires positioned along a portion or all of a longitudinal length of the expandable sheath and optional adhesion region used to facilitate in maintaining the expandable sheath in the unexpanded position;

FIG. 30 is a cross-sectional view of the expandable sheath of FIG. 27 and further optionally includes anti-kinking wires positioned along a portion or all of a longitudinal length of the expandable sheath and optional adhesion region used to facilitate in maintaining the expandable sheath in the unexpanded position;

FIG. 31 illustrates a frame member that can be positioned on the inner surface, outer surface and/or be partially or fully encapsulated in the body of the expandable sheath and fully encircles the expandable sheath and extends along a portion or complete longitudinal length of the expandable sheath;

FIG. 32 illustrates a frame member that can be positioned on the inner surface, outer surface and/or be partially or fully encapsulated in the body of the expandable sheath and is primarily or fully positioned in one or more of the thin bands of the expandable sheath and extends along a portion or complete longitudinal length of the expandable sheath;

FIGS. 33-36 illustrate a frame member that is formed of one or more pieces that can be positioned on the inner surface, outer surface and/or be partially or fully encapsulated in the body of the expandable sheath and fully encircles the expandable sheath and extends along a portion or complete longitudinal length of the expandable sheaths of FIGS. 1-30;

FIG. 37 illustrates a plurality of frame members that are connected together along a portion or complete longitudinal length of the expandable sheath; and

FIG. 38 illustrates a plurality of frame members that are spaced apart from one another along a longitudinal length of the expandable sheath.

### DESCRIPTION OF NON-LIMITING EMBODIMENTS OF THE DISCLOSURE

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Percentages of elements should be assumed to be percent by weight of the stated element, unless expressly stated otherwise.

Although the operations of exemplary embodiments of the disclosed method may be described in a particular, sequential order for convenient presentation, it should be understood that disclosed embodiments can encompass an order of operations other than the particular, sequential order disclosed. For example, operations described sequentially may in some cases be rearranged or performed concurrently. Further, descriptions and disclosures provided in association with one particular embodiment are not limited to that embodiment, and may be applied to any embodiment disclosed.

For the sake of simplicity, the attached figures may not show the various ways (readily discernable, based on this disclosure, by one of ordinary skill in the art) in which the disclosed system, method and apparatus can be used in combination with other systems, methods and apparatuses. Additionally, the description sometimes uses terms such as “produce” and “provide” to describe the disclosed method. These terms are abstractions of the actual operations that can be performed. The actual operations that correspond to these terms can vary depending on the particular implementation and are, based on this disclosure, readily discernible by one of ordinary skill in the art.

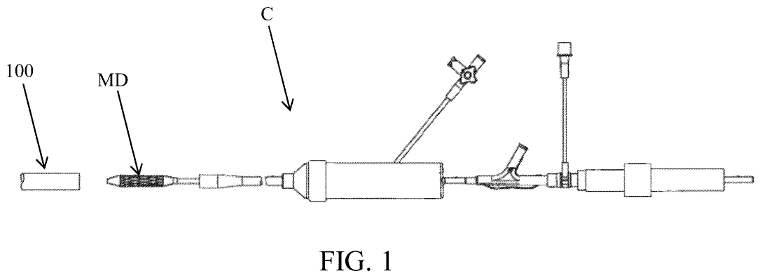

Referring now to FIG. 1, there is illustrate a portion of an expandable sheath 100 in accordance with the present disclosure that can be used with a representative delivery apparatus C for delivering a medical device (such as a TARV) MD to the heart of a patient; however, it will be appreciated that the medical device can be other types of devices (e.g., stent, balloon catheter, etc.). The delivery apparatus can optionally include a steerable guide catheter (also referred to as a flex catheter), a balloon catheter extending through the guide catheter, and an optional nose catheter extending through the balloon catheter. The guide catheter, the balloon catheter, and the nose catheter can be configured to slide longitudinally relative to each other to facilitate in the delivery and positioning of the medical device at a treatment site in the patient. Expandable sheath 100 can be inserted into a blood vessel such that one end of the expandable sheath is inserted into the blood vessel and the other end extends outside the body of a patient. Expandable sheath 100 can optionally include a valve (e.g., hemostasis valve) (not shown) at the proximal end of the expandable sheath. A portion of the delivery apparatus C that includes the medical device MD can be inserted into expandable sheath 100, and the medical device MD can then be delivered and implanted within a patient at the treatment site in the patient.

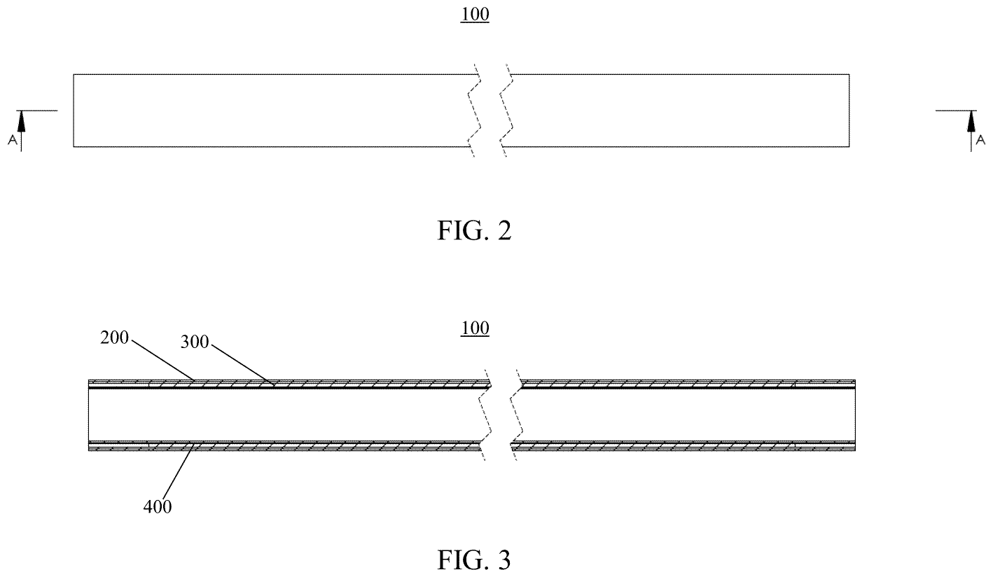

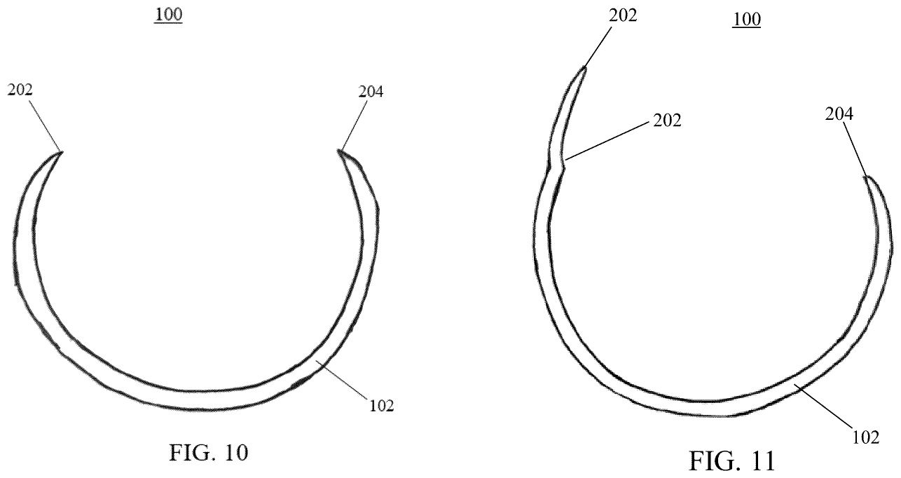

Referring now to FIGS. 2-38, several non-limiting embodiments of expandable sheath 100 in accordance with the present disclosure are illustrated. As illustrated in FIGS. 3 and 17-19, expandable sheath 100 can optionally include multiple layers.

Referring again to FIGS. 3-20, expandable sheath 100 includes an outer layer portion 200, a frame 300, and an inner layer portion 400. Outer layer portion 200, frame 300, and inner layer portion 400 facilitate in maintaining the shape and integrity of expandable sheath 100.

Inner layer portion 400 that can be formed of one or more polymer layers. The one or more polymer layers used to partially or fully form the inner layer portion can include polyethylene, polytetrafluoroethylene, polyimide, polyetheretherketone, polyurethane, nylon, polyether block amides, polyether block ester copolymer, thermoset silicone, latex, poly-isoprene rubbers, styrene ethylene butylene styrene, polyesters, fluoropolymers, polyvinyl chloride, polyolefin, and/or high-density polyethylene. In one non-limiting configuration, the inner layer portion is or includes polyethylene. When inner layer portion 400 includes two or more layers, the composition of the layers can be the same or different. In another non-limiting embodiment, the thickness of inner layer portion 400 is generally at least 0.001 inches, typically 0.015-0.006 inches. In another non-limiting embodiment, the inner layer portion is formed of a material that allows the outer diameter or outer cross-sectional area of expandable sheath 100 and the diameter or cross-sectional area of the interior passageway to expand and contract as the medical device is moved through expandable sheath 100 without damaging the material that forms inner layer portion 400.

The expandable sheath includes an outer layer portion 200 that can be formed of one or more polymer layers. The outer layer portion can be formed of the same or similar material as the inner layer portion. In one non-limiting embodiment, the one or more polymer layers used to partially or fully form the outer layer portion can include polytetrafluoroethylene (PTFE), polyimide, polyetheretherketone (PEEK), polyurethane, nylon, polyethylene, polyamide, styrenic block copolymer (SBC) (e.g., polystyrene blocks and rubber blocks [e.g., polybutadiene, polyisoprene or their hydrogenated equivalents]), polyether block amides, polyether block ester copolymer, thermoset silicone, latex, poly-isoprene rubbers, high density polyethylene (HDPE), block copolymers made up of polyamide blocks and polyether blocks (e.g., Pebax™, etc.), polyurethane resin, elastomers formed of block copolymers made up of rigid polyamide blocks and soft polyether blocks, or combinations thereof. In one non-limiting configuration, the outer layer portion is or includes polyether blocks (e.g., Pebax™, etc.). In another non-limiting embodiment, the thickness of outer layer portion 200 is generally at least 0.004 inches, typically 0.004-0.03 inches, and more typically 0.01-0.02 inches. In another non-limiting embodiment, the thickness of outer layer portion 200 is greater than the thickness of inner layer portion 400. In another non-limiting embodiment, the composition of the outer layer portion is different from the composition of the inner layer portion. In another non-limiting embodiment, the outer layer portion is formed of a material that allows the outer diameter or outer cross-sectional area of the expandable sheath and the diameter or cross-sectional area of the interior passageway to expand and contract as the medical device is moved through the expandable sheath without damaging the material that forms outer layer portion 200. The composition of the outer layer portion 200 can be the same or different from the composition of the inner layer portion 400.

Expandable sheath 100 includes a frame 300 that optionally includes a shape memory material. In one non-limiting embodiment, the majority (e.g., 60-99.99% and all values and ranges therebetween) or all of the frame is formed of a shape memory material. The shape memory material is or includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X=Zr, B, Cr, Gd) alloy, l) Cu—Al—Ni—Hf alloy, m) Cu—Sn alloy, n) Cu—Zn alloy, o) Cu—Zn—X (X=Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly (ethyleneglycol) dimethacrylate (tBA-co-PEGDMA) polymers, etc.), z) (meth) acrylate-based SMPs, aa) polyurethane-based SMPs, and/or bb) blends of polyurethane and polyvinylchloride-based SMPs. In one non-limiting configuration, frame 300 is partially or fully formed of nickel-titanium alloy.

The thickness of frame 300 of expandable sheath 100 is generally at least 0.002 inches, typically 0.002-0.03 inches, and more typically 0.004-0.012 inches. In another non-limiting embodiment, the thickness of the frame is greater than the thickness of inner layer portion 400. In another non-limiting embodiment, the thickness of frame 300 is equal to or less than the thickness of outer layer portion 200. In another non-limiting embodiment, the thickness of the frame is less than the thickness of the outer layer portion.

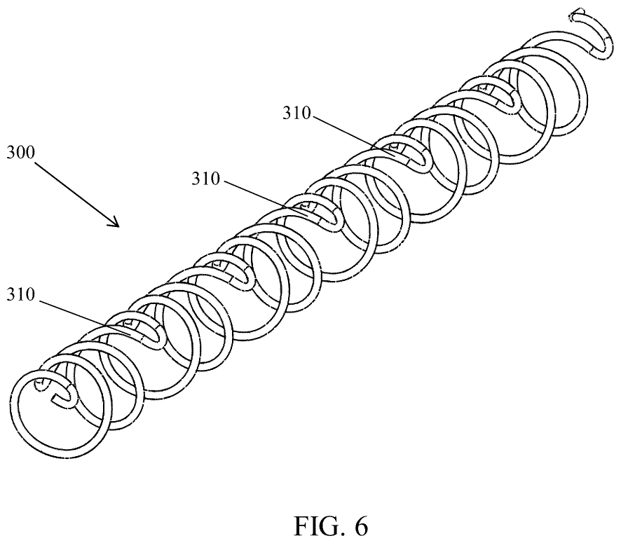

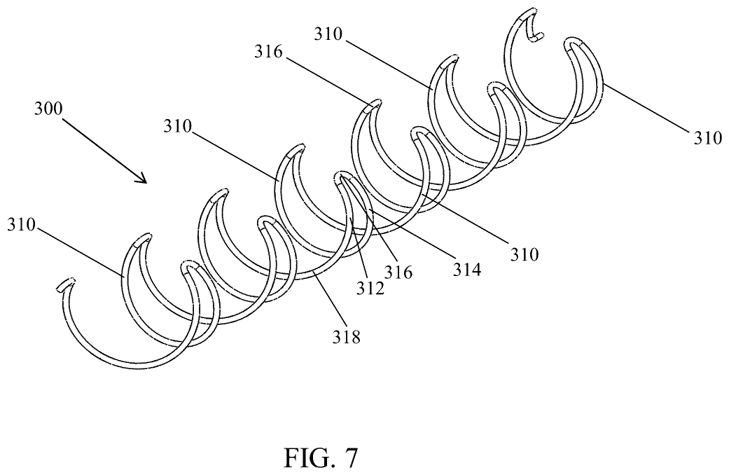

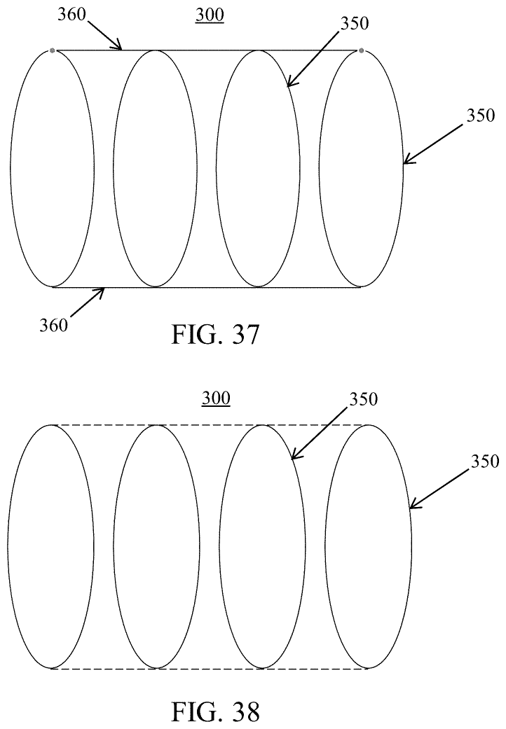

The frame 300 can extend along 10-100% (and all values and ranges therebetween) the longitudinal length of expandable sheath 100. The frame can be formed of a) a single piece of material that has the same size and/or shape along a longitudinal length of the frame (See e.g., FIGS. 6-7), b) a plurality of pieces that are that are positioned along the longitudinal axis of the expandable sheath and which are connected together (See e.g., FIG. 37), c) or be formed of a plurality of pieces that are positioned along the longitudinal axis of the expandable sheath and are spaced from one another along the longitudinal axis of the expandable sheath (See e.g., FIG. 38).

Frame 300 of the expandable sheath is formed of a material that allows the outer diameter or outer cross-sectional area of expandable sheath 100 and the diameter or cross-sectional area of the interior passageway to expand and contract as the medical device is moved through expandable sheath 100 without damaging the frame. In one non-limiting embodiment, the frame provides strength, structure and/or shape to expandable sheath 100.

FIG. 6 illustrates one non-limiting frame 300 in the contracted position, and FIG. 7 illustrates frame 300 of FIG. 6 in the expanded position.

As illustrated in FIGS. 6 and 7, frame 300 is formed of a single wire that has been bent to form multiple symmetrically oriented arm sections 310 along the longitudinal length of the frame. Arm sections 310 are illustrated as having generally the same shape and size.

Arm sections 310 are illustrated as overlapping one another along the longitudinal axis of the frame when the frame is in the expanded and/or contracted positions. Also, arm sections 310 are illustrated as not overlapping one another along the lateral axis of the frame.

As best illustrated in FIG. 7, arm sections 310 have a generally crescent shape. Each side of the crescent shape of arm section 310 is formed of two wire portions 312, 314 that are bent in a U-shape to form the tip portion 316. The U-shape of tip portion 316 has a radius of curvature that is 1.5-30 times less or smaller than a radius of curvature of the base portion 318 of arm section 310 when the frame is in the expanded position as illustrated in FIG. 7. As best illustrated in FIG. 7, except at the two ends of frame 300, base portion 318 is shared by two adjacently positioned arm sections 310.

As best illustrated in FIG. 7, tip portion 316 of arm sections 310 lies in a plane that is ±10° to the longitudinal axis of the frame (e.g., 0-3° to the longitudinal axis of the frame). Base portion 318 of arm sections 310 lies in a plane that is 65-88° to the longitudinal axis of the frame (e.g., 70-85°).

As illustrated in FIG. 6, tip portions 316 of arm sections 310 overlap one another along the longitudinal axis of the frame when the frame is in the contracted or unexpanded position.

As illustrated in FIG. 7, tip portions 316 of arm sections 310 do not overlap one another along the longitudinal axis of the frame when the frame is in the fully expanded position.

The pattern and shape of frame 300 enables the outer diameter or outer cross-sectional area of expandable sheath 100 and the diameter or cross-sectional area of the interior passageway to expand and contract as the medical device is moved through expandable sheath 100 without damaging frame 300. The frame 300 can be partially or fully formed of a shape memory material. The shape memory material used to at least partially form frame 300 facilitates in causing expandable sheath 100 to contract to its original or near original shape after being expanded as the medical device passes through expandable sheath 100.

The wire used to form of frame 300 can optionally have a generally constant size and cross-sectional area and shape, and frame 300 can be formed of a single piece of wire. Frame 300 is configured to facilitate in providing structure and strength to expandable sheath 100 and to also facilitate in causing expandable sheath 100 to move to the unexpanded position after expandable sheath 100 has been expanded.

Inner and outer layer portions 400, 200 are subjected to a connection processed to connect together outer layer portion 200 and inner layer portion 400. One non-limiting connection process can optionally be a reflow process to secure outer layer portion 200 and inner layer portion 400 by a heat bonding process (e.g., heat fusion process or heat melt process). As can be appreciated, additional or alternative connection processes can be used (e.g., adhesive, mechanical connection, crimping, etc.). As illustrated in FIGS. 3 and 17-20, when outer and inner layer portions 200, 400 are assembled together, frame 300 can be optionally positioned partially or fully between inner and outer layer portions 400, 200. As can be appreciated, frame 300 can be partially or fully positioned in inner layer portion 400 and/or outer layer portion 200. When inner and outer layer portions 400, 200 are secured together, frame 400 is also caused to be fixed in position between inner and outer layer portions 400, 200.

The inner surface of inner layer portion 400 can optionally include a lubrication coating and/or liner to facilitate in the movement of a medical device through expandable sheath 100. In one non-limiting embodiment, the inner surface of the inner layer portion has a coefficient of friction of no more about 0.1 (e.g., 0.0001-0.1 and all values and ranges therebetween).

The outer surface of outer layer portion 200 can optionally include a lubrication coating and/or liner to facilitate in the movement of expandable sheath 100 into and/or out of a body passageway. In one non-limiting embodiment, the outer surface of outer layer portion 300 has a coefficient of friction of no more about 0.1 (e.g., 0.0001-0.1 and all values and ranges therebetween).

The outer surface of outer layer portion 200 can optionally include a hydrophilic coating to facilitate in the insertion of expandable sheath 100 into and/or out of a body passageway. Non-limiting examples of hydrophilic coatings include the Harmony™ Advanced Lubricity Coatings and other Advanced Hydrophilic Coatings available from SurModics, Inc., Eden Prairie, Minn. and DSM medical coatings (available from Koninklijke DSM N.V, Heerlen, the Netherlands).

The inner diameter of cavity 500 through the longitudinal length of expandable sheath 100 prior to expandable sheath 100 being expanded can be 0.08-0.25 inches (and all values and ranges therebetween), and the maximum inner diameter of the cavity through the longitudinal length of expandable sheath 100 after expandable sheath 100 being expanded can be at least 0.15 inches. The wall thickness of expandable sheath 100 can be generally less than 0.1 inches, and typically less than 0.05 inches. The longitudinal length of expandable sheath 100 can be at least 3 inches and typically 3-40 inches (and all values and ranges therebetween).

The shape of expandable sheath 100 can optionally be tubular or cylindrical. The shape and size of the expandable sheath can be uniform along a majority (e.g., 60-99.99% and all values and ranges therebetween) or the full longitudinal length of the expandable sheath.

Expandable sheath 100 is configured to have a flexibility to allow expandable sheath 100 to be bent along the longitudinal axis of expandable sheath 100 so as to facilitate in the insertion of expandable sheath 100 into a body passageway.

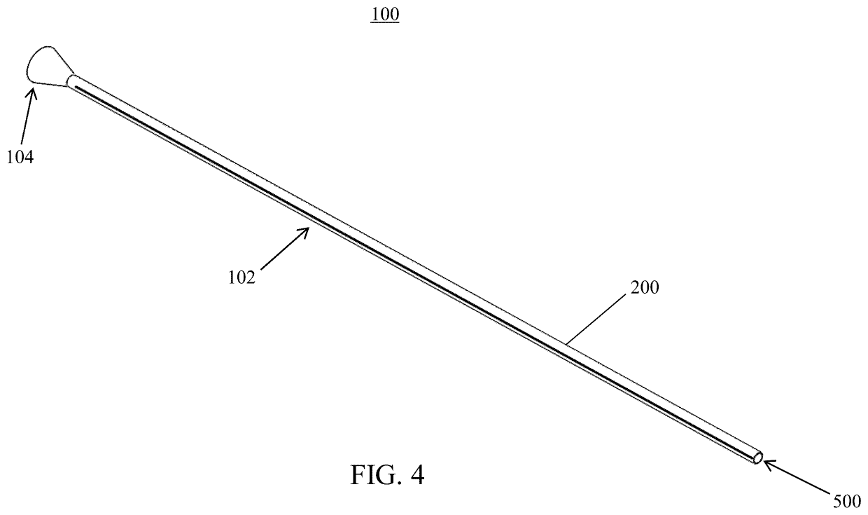

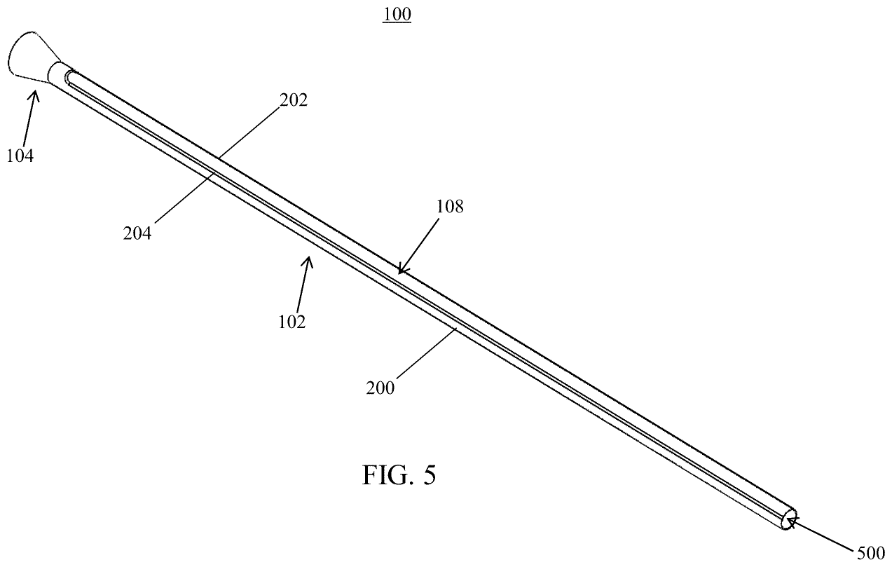

Referring now to FIGS. 4 and 5, expandable sheath 100 can optionally include a generally tubular-shaped portion 102 and an optional conical-shaped portion 104 that is connected to the front or proximal end of tubular-shaped portion 102. Tubular-shaped portion 102 generally constitutes 80-100% (and all values and ranges therebetween) of the longitudinal length of expandable sheath 100. When expandable sheath 100 includes conical-shaped portion 102, conical-shaped portion 102 includes an opening therethrough such that a medical device can be inserted into conical-shaped end portion 104 and into the cavity of tubular-shaped portion 102 of expandable sheath 100. The maximum cross-sectional area of the cavity of conical-shaped portion 104 is generally at least 1.5 times the cross-sectional area of the internal passageway or cavity of tubular-shaped portion 102 of expandable sheath 100 when in the collapsed or unexpanded position. Conical-shaped portion 104 can be formed of the same or different material as the polymer used to form the inner and outer layer portions 400, 200 of expandable sheath 100. Conical-shaped portion 104 can be connected to the front end of tubular-shaped portion 102 by a variety of mechanisms (e.g., adhesive, melted connection, friction connection, mechanical connection, solder, etc.). As illustrated in FIG. 5, the region of the tubular-shaped portion 102 that is at or near the conical-shaped portion 104 is not expandable. This non-expandable portion of the tubular-shaped portion 102, when used, constitutes 0.1-10% (and all values and ranges therebetween) of the longitudinal length of tubular-shaped portion 102. The non-expandable portion of the tubular-shaped portion 102, when used, can optionally have an inner diameter or cross-sectional area along a longitudinal length of a portion or all of the non-expandable portion that is greater than the inner diameter or cross-sectional area of the expandable portion of the tubular-shaped portion 102 when in the unexpanded position.

FIG. 4 illustrates tubular-shaped portion 102 of expandable sheath 100 in the unexpanded position along the full length of tubular-shaped portion 102. FIG. 5 illustrates tubular-shaped portion 102 of expandable sheath 100 in the expanded position along moth or all of the full length of tubular-shaped portion 102. As can be appreciated, when a medical device passes through cavity 500 of tubular-shaped portion 102, tubular-shaped portion 102 can be caused to expand to the expanded position along the full longitudinal length of tubular-shaped portion 102, or only expand to the expanded position in the region about the medical device and the remaining portions of tubular-shaped portion 102 remains in the unexpanded position. As illustrated in FIG. 5, edges 202, 204 of outer layer portion 200 are separated from one another to form a slot opening 108 in expandable sheath 100 when the expandable sheath 100 is in the expanded position. When expandable sheath 100 is in the non-expanded position as illustrated in FIG. 4, edges 202, 204 of outer layer portion 200 are in contact with one another and/or are positioned closely adjacent to one other.

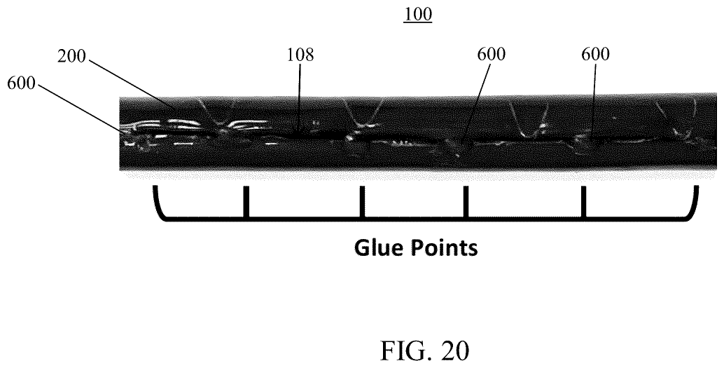

When the expandable sheath is first inserted into a body passageway and prior to a medical device being inserted through expandable sheath 100 that causes expansion of expandable sheath 100, edges 202, 204 can optionally be connected together by one or more breakable connection points 600 on a) outer layer portion 200 of expandable sheath 100 and/or b) outer layer portion 200 of expandable sheath 100 and optional lip 700 to maintain expandable sheath 100 in the collapsed or unexpanded position. The one or more breakable connection points 600 can be formed of a) an adhesive, b) melted portions, c) breakable stitching, d) hook and look fastener, and/or e) mechanical and/or friction connection. In one non-limiting embodiment, the expandable sheath 100 includes a single breakable connection point 600 that extends 5-100% (and all values and ranges therebetween) along a longitudinal length of edges 202, 204. In another non-limiting embodiment, the expandable sheath 100 includes a plurality of breakable connection points 600 that are spaced from one another and extend 5-99% (and all values and ranges therebetween) along a longitudinal length of edges 202, 204. The spacing between the plurality of breakable connection points 600 can be the same or different. The distance of spacing between the plurality of breakable connection points 600 is non-limiting. The length of each of the plurality of breakable connection points 600 is non-limiting, and the length of two or more breakable connection points 600 can be the same or different. In one non-limiting arrangement, one or more breakable connection points 600 are formed by a melted connection. When a plurality of breakable connection points 600 are located on expandable sheath 100, the plurality of breakable connection points 600 can be optionally spaced from one another at generally the same distance as illustrated in FIG. 20. The one or more breakable connection points are configured to break as a medical device passes through the cavity 500 of tubular-shaped portion 102 of expandable sheath 100 to enable tubular-shaped portion 102 to move from the unexpanded position to the expanded position.

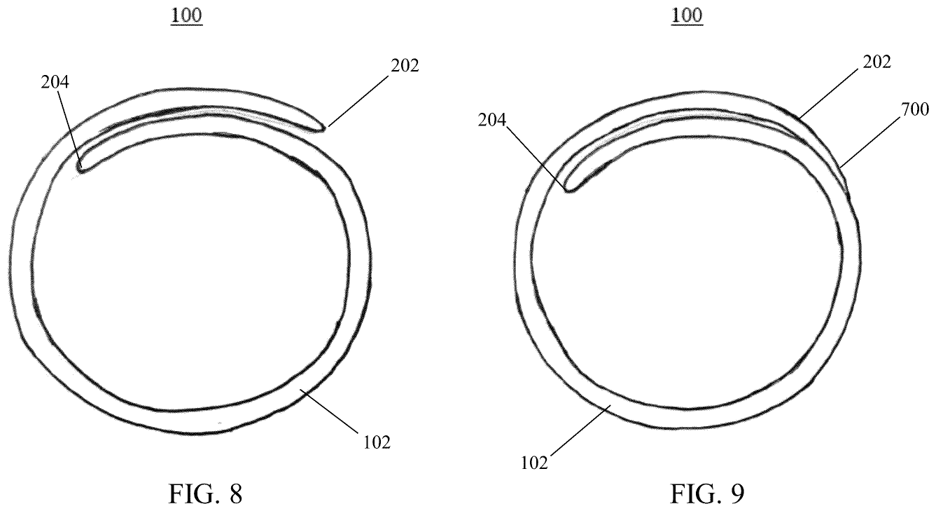

Referring now to FIGS. 8-16, there are illustrated front-end views of several non-limiting embodiments of the expandable sheath 100 in the expanded position and unexpanded positions.

FIGS. 8, 10, 12-14 and 16 illustrate expandable sheath 100 having edges 202, 204 and is absent a lip 700.

FIGS. 9, 11 and 15 illustrate expandable sheath 100 having edges 202, 204 and have a lip 700. Optional lip 700 (when used) can optionally have a tapered thickness. Optional lip 700 can be used to ensure slot opening 108 is closed when expandable sheath 100 is in the unexpanded position.

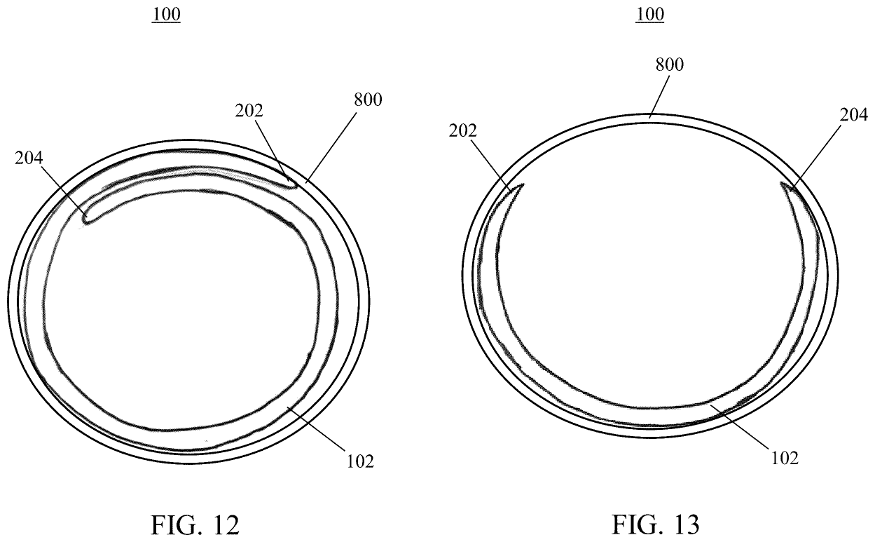

FIGS. 12, 13 and 16 illustrate the expandable sheath 100 having an optional outer expandable cover 800. Expandable cover 800 can be formed of an elastomeric or flexible polymer (e.g., natural rubbers, styrene-butadiene block copolymers, polyisoprene, polybutadiene, ethylene propylene rubber, ethylene propylene diene rubber, silicone elastomers, fluoroelastomers, polyurethane elastomers, nitrile rubbers, etc.). The expandable cover 800 can optionally be formed of or include a shape memory material. The expandable cover 800, when used, is configured to facilitate in a) maintaining the expandable sheath 100 in the unexpanded position in the regions where the medical device is not located in the expandable sheath 100, and/or b) facilitating in causing the expandable sheath 100 to move from the expanded positioned to at or close to the unexpanded position after a medical device has passed through a portion or all of the expandable sheath 100. The thickness of the expandable cover 800 is non-limiting. One or more expandable covers 800 can be positioned along the longitudinal length of portion 102. In one non-limiting embodiment, the one or more expandable covers 800 are positioned along 5-100% (and all values and ranges therebetween) of the longitudinal length of portion 102. The expandable cover 800 can be sized and configured to apply a compression force toward the central axis of the expandable sheath 100 when the expandable sheath 100 is in the unexpanded position, the partially expanded position and/or the fully expanded position. As illustrated in FIGS. 12 and 16, the inner surface of the expandable cover 800 can be sized and configured to contact at least 50% (e.g., 50-100% an all values and ranges therebetween) of the outer surface of the portion 102 when the expandable sheath 100 is in the unexpanded position. The thickness of the expandable cover 800 can be uniform about the perimeter of the expandable cover 800; however, this is not required. The thickness of the expandable cover 800 is non-limiting (e.g., 0.1-5 mm and all values and ranges therebetween).

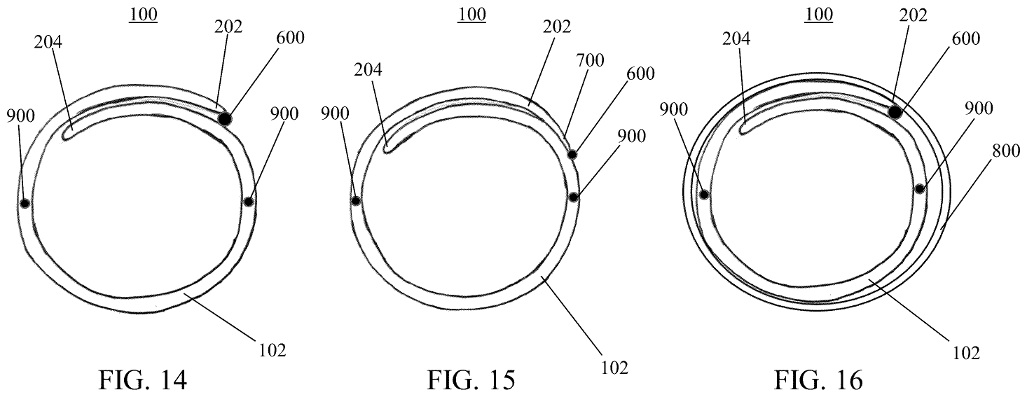

Referring now to FIGS. 14-16, the expandable sheath 100 optionally includes breakable connection points 600.

FIG. 15 illustrates the expandable sheath 100 optionally including lip 700. FIG. 16 illustrates the expandable sheath 100 optionally including expandable cover 800.

FIGS. 14-16 illustrate illustrates the expandable sheath 100 optionally including one or more anti-kinking members 900 that are positioned along a portion or all of the longitudinal length of portion 102. The number of anti-kinking members 900, when used, is non-limiting. As illustrated in FIGS. 14-16, the expandable sheath 100 includes two anti-kinking members 900 that are spaced about the perimeter of the expandable sheath 100 and which are positioned generally diametrically apart from one another about the central longitudinal axis of portion 102 when expandable sheath 100 is in the unexpanded position; however, one anti-kinking member 900 or more than two anti-kinking members 900; that the spacing between the two or more anti-kinking members 900 about the perimeter of the expandable sheath 100 can be such that there is equal or non-equal spaced between adjacently positioned anti-kinking members 900. In one non-limiting embodiment, one or more of the anti-kinking members 900 is partially or fully formed of a shape memory material. In another non-limiting embodiment, one or more of the anti-kinking members 900 extends 5-100% (and all values and ranges therebetween) the longitudinal length of the portion 102. In another non-limiting embodiment, one or more of the anti-kinking members 900 is spaced from the inner and/or outer surface of the expandable sheath 100. In another non-limiting embodiment, one or more of the anti-kinking members 900 is a generally cylindrical member that has a diameter that is less than a thickness between the inner and/or outer surface of the expandable sheath 100. Each the anti-kinking members 900 that is positioned along the longitudinal length of the expandable sheath 100 can be formed of a single piece of material or be formed of a multiple piece that are spaced from one another.

The one or more anti-kinking members 900, when used, facilitate in inhibiting or preventing portion 102 from a) overly bending or kinking when being moved within a body passageway, thereby facilitating in inhibiting or preventing damage to portion 102 during use of expandable sheath 100, and/or b) causing one or more regions of portion 102 to partially or fully re-straighten after such one or more regions of portion 102 have moved past a non-linear portion of the body passageway as portion 102 is moved in the body passageway.

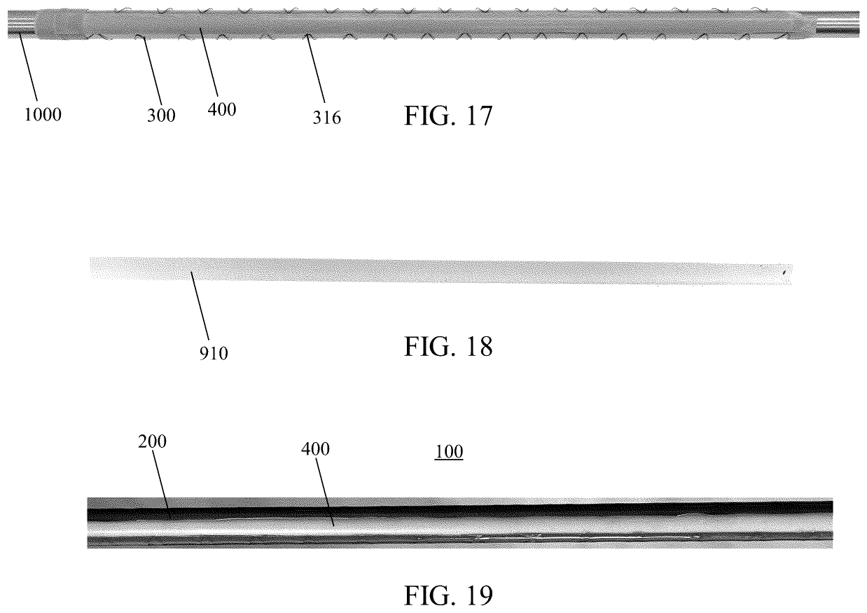

Referring now to FIGS. 17-20, a non-limiting method is partially illustrated for forming expandable sheath 100. As illustrated in FIG. 17, a mandrel or forming rod 1000 is provided. The diameter of mandrel or forming rod 1000 is greater than the diameter of cavity 500 of tubular-shaped portion 102 when tubular-shaped portion 102 is in the unexpanded position. In one non-limiting arrangement, the diameter of mandrel or forming rod 1000 is 1.5-10 times greater (and all values and ranges therebetween) the diameter of cavity 500 of tubular-shaped portion 102 when tubular-shaped portion 102 is in the unexpanded position. The material used to form mandrel or forming rod 1000 is non-limiting (e.g., metal, plastic, glass, ceramic, wood, composition material, etc.). Generally, the material used to form mandrel or forming rod 1000 has a greater melting point than the material used to form inner and outer layer portions 400, 200 of expandable sheath 100. The shape and diameter of mandrel or forming rod 1000 along 50-100% (and all values and ranges therebetween) is generally constant.

Referring again to FIG. 17, inner layer portion 400 is positioned about at least a portion of the outer surface of mandrel or forming rod 1000 or non-stick sheath 910. The non-stick-sheath 910, when used, inhibit or prevents the inner layer portion 400 from sticking to mandrel or forming rod 1000. In one non-limiting arrangement, inner layer portion 400 has a thickness of 0.001-0.003 inches and is formed of PTFE.

After inner layer portion 400 is positioned about at least a portion of the outer surface of mandrel or forming rod 1000, frame 300 is positioned about the inner layer portion 400. Frame 300 can be formed of a shape memory material (e.g., Nitinol™, etc.). When frame 300 is formed, frame 300 is generally heated set in the collapsed position so frame 300 that frame 300 will want to return to the collapsed position after being expanded. As illustrated in FIG. 17, frame 300 is in the full or partial expanded position when placed about inner layer portion 400.

After frame 300 is positioned about inner layer portion 400, the portion of inner layer portion 400 that is located between U-shaped tip portions 316 of expanded frame 300 can be optionally removed.

Referring now to FIG. 19, outer layer portion 200 is positioned about frame 300. The outer layer portion has a longitudinal slot or opening. Edges 202, 204 of outer layer portion 200 are illustrated as being lined-up with the ends of U-shaped tip portions 316 of expanded frame 300. In one non-limiting configuration, outer layer portion 200 can be formed of Pebax™ and has a thickness that is greater than a thickness of inner layer portion 400 and the thickness of frame 300.

Once outer layer portion 200 is properly positioned on frame 300, a strip of plastic tape 1100 (e.g., PTFE tape, etc.) can optionally be positioned between edges 202, 204 of outer layer portion 200. The plastic tape 1100 (when used) facilitates in inhibiting or prevent the outer layer portion to flow into the region between U-shaped tip portions 316 of expanded frame 300 when inner layer portion 400 and outer layer portion 200 are heated and connected together.

Once outer layer portion 200 is properly positioned on frame 300, inner layer portion 400 and outer layer portion 200 are connected together. Generally, inner layer portion 400 and outer layer portion 200 are connected together by a heat or reflow process. Such heat or reflow process can occur by placing mandrel or forming rod 1000 that includes inner layer portion 400, frame 300, outer layer portion 200 and optional plastic tape 1100 into a heating oven.

After inner layer portion 400 and outer layer portion 200 are connected together, optional plastic tape 1100 (when used) can be removed, and thereafter mandrel or forming rod 1000 is removed. Once mandrel or forming rod 1000 is removed, frame 300 will cause tubular-shaped portion 102 to contact to the expanded position. Thereafter, optional breakable connection points 600 on outer layer portion 200 of expandable sheath 100 and/or on outer layer portion 200 of expandable sheath 100 and optional lip 700 can be formed to maintain expandable sheath 100 in the collapsed or unexpanded position. The optional conical front-end portion 104 can also be connected to the front end of tubular portion 102.

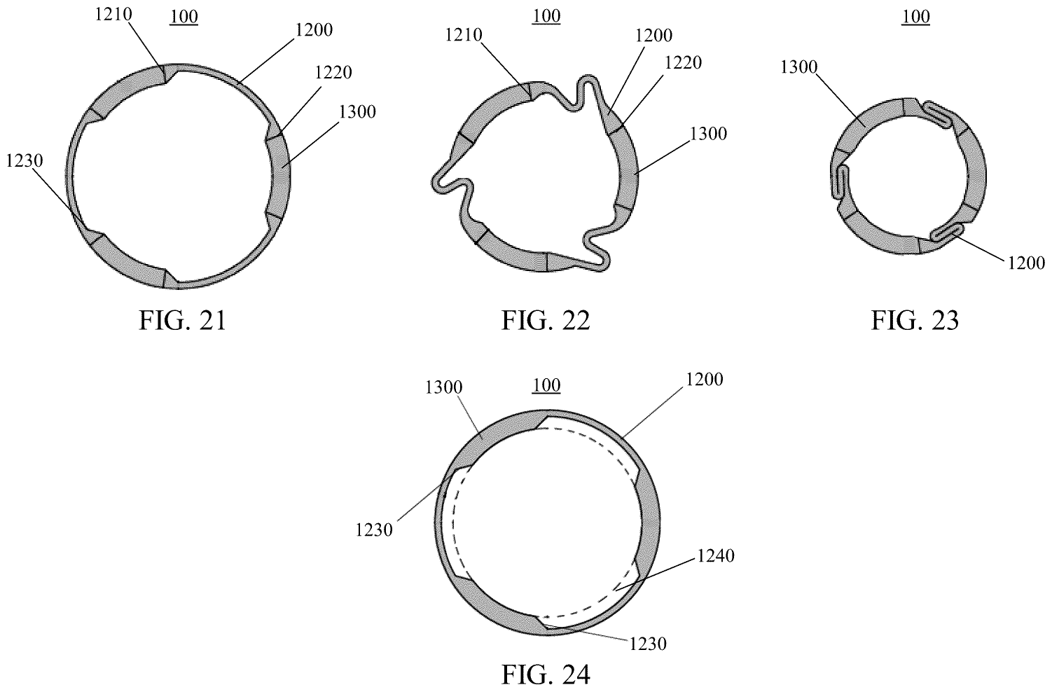

Referring now to FIGS. 21-36, another non-limiting expandable sheath 100 is illustrated. FIGS. 21-27 illustrate a front-end view or a front face of the expandable sheath 100. The shape of front face or the front-end view of the expandable sheath 100 extends 20-100% (and all values and ranges therebetween) the longitudinal length of the expandable sheath 100. The longitudinal length of the expandable sheath can be at least 3 inches and typically 3-40 inches (and all values and ranges therebetween).

The expandable sheath 100 can have a wide variety of inner and outer diameters. The expandable sheath 100 can be configured to expand to an expanded outer diameter that is from about 10% greater than the original unexpanded outer diameter to about 300% greater than the original unexpanded outer diameter (and all values and ranges therebetween).

The expandable sheath 100 illustrated in FIGS. 21-27 can formed of a) a uniform single material, b) formed of multiple material, and/or c) include a frame structure 300.

The expandable sheath 100 illustrated in FIGS. 21-27 can formed of a single piece of material as illustrated in FIG. 24, or can be formed of multiple pieces of material that are connected together as illustrated in FIGS. 21-23 and 25-27.

In one non-limiting embodiment, the expandable sheath 100 illustrated in FIG. 24 is formed of a single polymer or a single polymer mixture. As can be appreciated, the expandable sheaths 100 illustrated in FIGS. 21-23 and 25-27 can also be formed of single polymer or a single polymer mixture.

In one specific non-limiting embodiment, the expandable sheath illustrated in FIG. 24 is formed of styrenic block copolymer (SBC) or high-density polyethylene (HDPE). As can be appreciated, other polymers can be used. In another specific non-limiting embodiment, the expandable sheath illustrated in FIG. 24 is formed of a metal or metal alloy. In another specific non-limiting embodiment, the expandable sheath illustrated in FIG. 24 is formed of shape memory material (e.g., shape memory metal alloy, shape memory polymer, etc.).

In another non-limiting embodiment, the expandable sheath illustrated in 21-23 and 25-27 is formed of two different materials (e.g., two different polymers, two different polymer mixtures, two different metal material, polymer material and a metal material, etc.). In one non-limiting embodiment, the expandable sheath is partially or fully formed of a polymer material and wherein the expandable sheath 100 includes at least two polymers, and wherein a portion of the expandable sheath is fully or partially formed of a polymer that forms a generally rigid structure 1300 and another flexible portion 1200 of the expandable sheath that is connected to the generally rigid structure 1300. The flexible portion 1200 can be partially or fully formed a flexible or elastomeric polymer (e.g., natural rubbers, styrene-butadiene block copolymers, polyisoprene, polybutadiene, ethylene propylene rubber, ethylene propylene diene rubber, silicone elastomers, fluoroelastomers, polyurethane elastomers, nitrile rubbers, etc.), and wherein the durometer of the flexible or elastomeric polymer that is used to partially or fully form the flexible portion 1200 is less (e.g., 1.2-2000 times less and all values and ranges therebetween) than the durometer of the polymer used to partially or fully form the generally rigid structure 1300. In another non-limiting embodiment, the generally rigid structure 1300 of the expandable sheath 100 can be configured to generally maintain its shape and size (e.g., maintain is original shape and/or size 0-10% [and all values and ranges therebetween] when the expandable sheath 100 is in the expanded and unexpanded positions) when the expandable sheath 100 expands and/or contracts between the first outer diameter or first outer cross-sectional area and the second outer diameter or second outer cross-sectional area. In another non-limiting embodiment, the flexible portion 1200 the expandable sheath 100 is configured to change its shape and/or size when the expandable sheath 100 expands and/or contracts between the first outer diameter or first outer cross-sectional area and the second outer diameter or second outer cross-sectional area.

In another non-limiting embodiment, the expandable sheath 100 includes one or more flexible portions 1200 and one or more generally rigid structures 1300. As illustrated in FIGS. 21-27, the expandable sheath 100 includes three flexible portions 1200 and three generally rigid structures 1300, and wherein each of the flexible portions 1200 is positioned between and connected to a generally rigid structure 1300.

In another non-limiting embodiment, one or more of the flexible portions 1200 is partially or fully formed of a shape memory material. Non-limiting shape member material includes a) copper-aluminum-nickel alloy, b) nickel-titanium alloy, c) zinc-copper-gold-iron alloy, d) Fe—Mn—Si alloy, e) Cu—Zn—Al alloy, f) Cu—Al—Ni alloy, g) Ag—Cd alloy, h) Au—Cd alloy, i) Co—Ni—Al alloy, j) Co—Ni—Ga alloy, k) Cu—Al—Be—X (X=Zr, B, Cr, Gd) alloy, l) Cu—Al—Ni—Hf alloy, m) Cu—Sn alloy, n) Cu—Zn alloy, o) Cu—Zn—X (X=Si, Al, Sn) alloy, p) Fe—Pt alloy, q) Mn—Cu alloy, r) Ni—Fe—Ga alloy, s) Ni—Ti—Hf alloy, t) Ni—Ti—Pd alloy, u) Ni—Mn—Ga alloy, v) Ni—Mn—Ga—Cu alloy, w) Ni—Mn—Ga—Co alloy, x) Ti—Nb alloy, y) polyacrylate-based SMPs (e.g., t-butylacrylate-co-poly (ethyleneglycol) dimethacrylate (tBA-co-PEGDMA) polymers, etc.), z) (meth) acrylate-based SMPs, aa) polyurethane-based SMPs, and/or bb) blends of polyurethane and polyvinylchloride-based SMPs. As defined herein, the flexible or elastomeric polymer is not a shape memory material. In another non-limiting embodiment, when the shape memory material is included in one or more of the flexible portions 1200, the shape memory material can include 5-100 wt. % (and all values and ranges therebetween) of each of the flexible portions 1200 that are formed of or include the shape memory material. When the flexible portion 1200 is not fully formed of the shape memory material, the flexible or elastomeric polymer can partially or fully coat the outer surface of the shape memory material. In another non-limiting embodiment, the generally rigid structure 1300 can optionally include a shape memory material. When the shape memory material is included in the one or more generally rigid structures 1300, the shape memory material can include 1-60 wt. % (and all values and ranges therebetween) of each of the generally rigid structures 1300 that include the shape memory material. In another non-limiting arrangement, one or more of the flexible portions 1200 and/or one or more of the generally rigid structures 1300 includes a shape memory material that is coated with a polymer material. In another non-limiting configuration, one or more of the flexible portions 1200 and/or one or more of the generally rigid structures 1300 includes a shape memory material that is partially or fully encapsulated in a non-shape memory polymer layer, or the shape memory material is partially or fully encapsulated in two or more layers of a non-shape memory polymer layer, wherein the polymer layers can be formed of the same or different polymer material.

In one specific non-limiting embodiment, the generally rigid structures or thick bands 1300 are partially or fully formed of high-density polyethylene (HDPE) and the flexible portions or thin bands 1200 are partially or fully formed of styrenic block copolymer (SBC). As can be appreciated, other polymers can be used.

As illustrated in FIGS. 21-23 and 25-27, the generally rigid structures or thick bands 1300 are formed of one piece of material and the flexible portions or thin bands 1200 are formed of another piece of material and wherein the generally rigid structures or thick bands 1300 and the flexible portions or thin bands 1200 are connected together (e.g., adhesive connection, melted seam connection, etc.) at a connection location 1210, 1220. In such an arrangement, the material used to form the flexible portions or thin bands 1200 can be the same or different from the material used to form the generally rigid structures or thick bands 1300.

In another non-limiting embodiment, the expandable sheath 100 illustrated in FIGS. 21-27 can be partially or fully formed by an extrusion process. In one non-limiting process, the expandable sheath 100 is formed by an extrusion process while the extruded expandable sheath 100 is in the fully expanded orientation as illustrated in FIGS. 21 and 24.

FIGS. 22, 24 and 23 illustrate the expandable sheath 100 in the fully expanded orientation. FIGS. 22 and 26 illustrate the expandable sheath 100 in the partially folded configuration. FIGS. 23 and 27 illustrate the expandable sheath 100 in the fully folded position or fully contracted orientation.

When the expandable sheath 100 is in the fully contracted orientation, the diameter of the inner cavity is about 0.1-0.25 inches (and all values and ranges therebetween, 0.131 inches, etc.) and the outer diameter is about 0.105-0.27 inches (and all values and ranges therebetween, 0.171 inches, etc.). When the expandable sheath is in the fully expanded orientation, the diameter of the inner cavity is about 0.15-0.3 inches (and all values and ranges therebetween, 0.206 inches, etc.) and the outer diameter is about 0.155-0.35 inches (and all values and ranges therebetween, 0.246 inches, etc.). As can be appreciated, these dimensions are non-limiting and are only representative of a single non-limiting embodiment of the disclosure.

As illustrated in FIGS. 21-27, the generally rigid structures or thick bands 1300 are generally not foldable or are non-foldable so that no portion of the generally rigid structures or thick bands 1300 overlaps another portion of the generally rigid structures or thick bands 1300 when the expandable sheath 100 moves between the fully expanded orientation and the fully contracted orientation. In one non-limiting arrangement, one, a plurality or all of the generally rigid structures or thick bands 1300 maintain the same or substantially the same shape along the longitudinal axis of the expandable sheath 100 as the expandable sheath 100 moves between the fully expanded orientation and the fully contracted orientation.

As illustrated in FIGS. 21-27, the flexible portions or thin bands 1200 are foldable. The foldability of the flexible portions or thin bands 1200 can be at least partially the result of 1) the flexible portions or thin bands 1200 being formed of a different material and/or a different number of layers from the generally rigid structures or thick bands 1300, b) the flexible portions or thin bands 1200 having a smaller thickness than the generally rigid structures or thick bands 1300, c) the flexible portions or thin bands 1200 having a different shape from the generally rigid structures or thick bands 1300, and/or d) the flexible portions or thin bands 1200 are processed differently (e.g., different heat treatments, different extrusion parameters, etc.) from the generally rigid structures or thick bands 1300. Although the generally rigid structures or thick bands 1300 have a rigidity that inhibits or prevents the generally rigid structures or thick bands 1300 from folding over itself as the expandable sheath 100 moves between the fully expanded orientation and the fully contracted orientation, both the generally rigid structures or thick bands 1300 and flexible portions or thin bands 1200 can have a flexibility to allow the expandable sheath 100 to be bent along the longitudinal axis of the expandable sheath 100 so as to facilitate in the insertion of the expandable sheath 100 into a body passageway.

As illustrated in FIGS. 21-27, the expandable sheath 100 is illustrated as including three flexible portions or thin bands 1200 and three generally rigid structures or thick bands 1300. Generally, the expandable sheath 100 includes a plurality of flexible portions or thin bands 1200 (e.g., 2-20 and all values and ranges therebetween) and a plurality of generally rigid structures or thick bands 1300 (e.g., 2-20 and all values and ranges therebetween). Generally, two or more or all of the flexible portions or thin bands 1200 have the same size, shape, configuration, and/or composition; however, this is not required. Generally, two or more or all of the generally rigid structures or thick bands 1300 have the same size, shape, configuration, and/or composition; however, this is not required. As illustrated in FIGS. 21-27, the size, shape and configuration of all the flexible portions or thin bands 1200 are the same, and the size, shape and configuration of all the generally rigid structures or thick bands 1300 are the same. In one non-limiting embodiment, thickness of the generally rigid structures or thick bands 1300 is being about 0.02-0.06 inches (and all values and ranges therebetween, 0.04 inches, etc.) thick; however, other thickness can be used. Generally, the thickness of the flexible portions or thin bands 1200 is generally about 10-80% (and all values and ranges therebetween) of the thickness of the generally rigid structures or thick bands 1300. Generally, the thickness of the flexible portions or thin bands 1200 is 30-50% the thickness of the generally rigid structures or thick bands 1300. Each end of a generally rigid structures or thick bands 1300 is connected to or merges with an end of an adjacently positioned flexible portions or thin bands 1200.

The total volume of the generally rigid structures or thick bands 1300 is about 30-80 vol. % (and all values and ranges therebetween) of the expandable sheath 100. In one non-limiting embodiment, the total volume of the generally rigid structures or thick bands 1300 is about 55-75 vol. % of the expandable sheath 100.

The total outer surface of the generally rigid structures or thick bands 1300 constitutes about 30-80% (and all values and ranges therebetween) of the outer circumference of the expandable sheath 100 when the expandable sheath 100 is in the fully expanded orientation as illustrated in FIGS. 21, 24 and 25. In one non-limiting embodiment, total outer surface of the generally rigid structures or thick bands 1300 constitutes about 35-48% of the outer circumference of the expandable sheath 100 when the expandable sheath is in the fully expanded orientation.

As best illustrated in FIGS. 21, 24 and 25 when the expandable sheath 100 is in the fully expanded position, the inner surface of each end portion of each of the flexible portions or thin bands 1200 is illustrated as having a tapered region 1230 to transition to the thickness of the generally rigid structures or thick bands 1300; however, this is not required. As illustrated in FIGS. 21, 24 and 25, the thickness of the tapered region 1230 is thickest at the point where the tapered region 1230 terminates at generally rigid structures or thick bands 1300.

Each of the tapered regions 1230 are illustrated to have the same size, shape and configuration; however, this is not required. The length of each taper regions 1230, when used, is generally 2-40% (and all values and ranges therebetween) of the circumferential length of the flexible portions or thin bands 1200. The thickness of each of the flexible portions or thin bands 1200 is illustrated as being generally uniform along 80-100% (and all values and ranges therebetween) of the circumferential length of the thin band that is absent the taper regions 1230; however, this is not required. Although the tapered regions 1230 are illustrated as forming a portion of the flexible portions or thin bands 1200, it can be appreciated that the tapered regions 1230 could alternatively form a portion of generally rigid structures or thick bands 1300.

As illustrated in FIGS. 21, 24 and 25, the outer cross-sectional profile of the expandable sheath 100 in the fully expanded orientation is generally circular. In one non-limiting embodiment, outer surface of one or more or all of the flexible portions or thin bands 1200 and one or more or all of the generally rigid structures or thick bands 1300 have the same radius of curvature or a similar radius of curvature (e.g., radius of curvature of the outer surface of the flexible portions or thin bands 1200 is 0.01-5% [and all values and ranges therebetween) of a radius of curvature of the outer surface of the generally rigid structures or thick bands 1300) along 70-100% (and all values and ranges therebetween) of the outer surface or perimeter of the flexible portions or thin bands 1200 and the generally rigid structures or thick bands 1300 when the expandable sheath 100 in the fully expanded orientation. As illustrated in FIG. 24, the radius of curvature of the inner surface of each of the generally rigid structures or thick bands 1300 when the expandable sheath 100 in the fully expanded orientation is the same or generally the same (e.g., 0.01-5% and all values and ranges therebetween of a radius of curvature of the inner surface of each of the generally rigid structures or thick bands 1300) when the expandable sheath 100 in the fully expanded orientation, and/or the radius of curvature of the inner surface of each of the flexible portions or thin bands 1200 that does not include the tapered regions 1230 when the expandable sheath 100 in the fully expanded orientation is the same or generally the same (e.g., 0.01-5% and all values and ranges therebetween of a radius of curvature of the inner surface of each of the flexible portions or thin bands 1200 that does not include the tapered regions 1230) when the expandable sheath 100 in the fully expanded orientation. The dashed lines illustrated in FIG. 24 illustrated a generally circular profile is formed by the inner surface of each of the flexible portions or thin bands 1200 that does not include the tapered regions 1230 when the expandable sheath 100 in the fully expanded orientation. Likewise, a generally circular profile is formed by the inner surface of each of the generally rigid structures or thick bands 1300 when the expandable sheath 100 in the fully expanded orientation.

Referring now to FIG. 28, one or more of the flexible portions or thin bands 1200 and/or one or more of the generally rigid structures or thick bands 1300 can optionally be formed of two or more layers, and wherein the layers can be the same or different materials. For example, FIG. 28 illustrates a portion of one of flexible portions or thin bands 1200 can be formed of outer layers 1240, 1260 and coated on or otherwise secure to the outer surface 1270 of inner layer 1250. As can be FIG. 28 can alternatively illustrate a portion of generally rigid structures or thick bands 1300. In one non-limiting embodiment, inner layer 1250 can optionally be formed of a shape memory material and outer layers 1240, 1260 can be formed or a polymer material that has a different composition from inner layer 1250. In another non-limiting embodiment, the inner layer 1250 can optionally represent a portion of frame 300 that is optionally positioned in a portion of one or more of flexible portions or thin bands 1200 and/or generally rigid structures or thick bands 1300. The thickness of inner layer 1250 is illustrated as being greater than a thickness of either of outer layers 1240, 1260; however, this is not required. The thickness of each of outer layers 1240, 1260 is illustrated as being generally the same; however, this is not required. In one non-limiting embodiment, inner layer 1250 is formed of a shape memory material, and one or both of outer layers 1240, 1260 is formed of a flexible or elastomeric polymer.

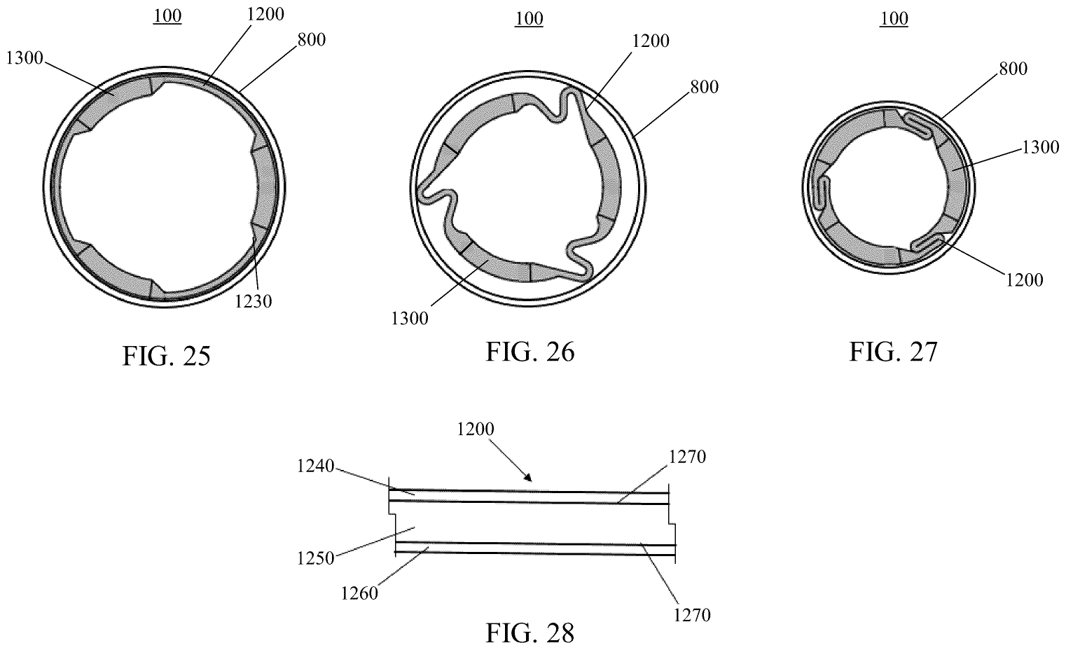

Referring now to FIGS. 25-27 and 30, the expandable sheath 100 can optionally include an expandable cover 800. The shape, size, thickness, material and/or function of the expandable cover 800 can be the same or similar to the expandable cover 800 as described above with regard to FIGS. 12, 13 and 16.

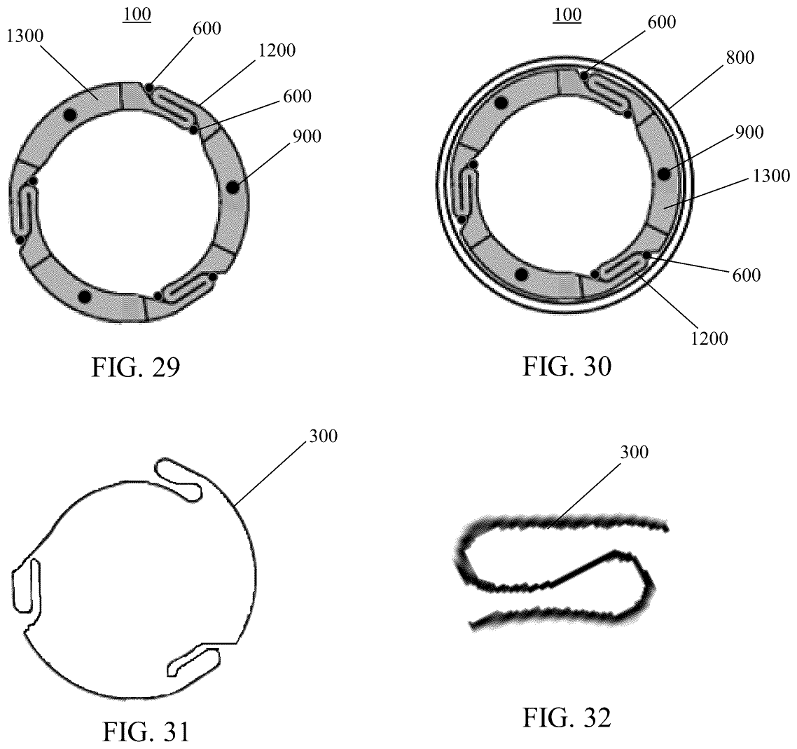

Referring now to FIGS. 29 and 30, the expandable sheath 100 can optionally include one or more anti-kinking members 900. The shape, size, thickness, material and/or function of the anti-kinking members 900 can be the same or similar to the anti-kinking members 900 as described above with regard to FIGS. 14-16.

Referring now to FIGS. 29 and 30, the expandable sheath 100 can optionally include one or more breakable connection points 600. The shape, size, thickness, material and/or function of the breakable connection points 600 can be the same or similar to the breakable connection points 600 as described above with regard to FIGS. 14-16.

Referring now to FIGS. 31-36, the expandable sheath 100 can optionally include one or more frames 300. The frame 300 may or may not be fully or partially formed of a shape memory material. The shape of the frame 300, when used is non-limiting. The frame 300 can extend 5-100% (and all values and ranges therebetween) the longitudinal length of the expandable sheath 100. The frame 300 can a) be formed of a single piece of material that has a uniform shape along 10-100% (and all values and ranges therebetween) the longitudinal length of the frame member 300, b) be formed of plurality of frame sections that are spaced apart from one another along the longitudinal length of the frame 300, and wherein two or more of the frame sections are not connected together by another portion of the frame 300, or c) be formed of plurality of frame sections that are spaced apart from one another along the longitudinal length of the frame 300, and wherein two or more of the frame sections are connected together by another portion of the frame 300 (e.g., one or more spine members of the frame 300, etc.). FIG. 37 illustrates a plurality of frame sections 350 that are spaced apart from one another along the longitudinal length of the frame 300, and wherein two or more of the frame sections 350 that are spaced apart from one another and are connected together by a spine member 360 of the frame 300. The spacing between three or more adjacently positioned spine members 360 along the longitudinal length of the frame 300 can be the same or different. As can be appreciated, the one or more spine members 360 can optionally function also as an anti-kinking member 900.

FIG. 38 illustrates a plurality of frame sections 350 that are spaced apart from one another along the longitudinal length of the frame 300, and wherein two or more of the frame sections 350 that are spaced apart from one another and are not connected together. The spacing between three or more adjacently positioned spine members 360 along the longitudinal length of the frame 300 can be the same or different. The cumulative longitudinal length of the plurality of frame sections 350 along the longitudinal length of the expandable sheath 100 is 5-99% (and all values and ranges therebetween) of the longitudinal length of the expandable sheath 100.

Referring again to FIGS. 31-36, various non-limiting cross-sections of the frame 300 or a frame section 350 are illustrated.

FIG. 31 illustrates a cross-section of the frame 300 or a frame section 350 that is formed of a single piece about the perimeter of the frame 300 or a frame section 350. The shape of the frame 300 or a frame section 350 is the same or similar to the cross-sectional shape of unexpanded expandable sheath 100 as illustrated in FIGS. 23, 27, 29, and 30.

FIG. 32 illustrates a cross-section of the frame 300 or a frame section 350 that is formed of a single piece. The shape of the frame 300 or a frame section 350 is the same or similar to the cross-sectional shape of one of flexible portions or thin bands 1200 when the expandable sheath 100 is in the unexpanded position as illustrated in FIGS. 23, 27, 29, and 30.

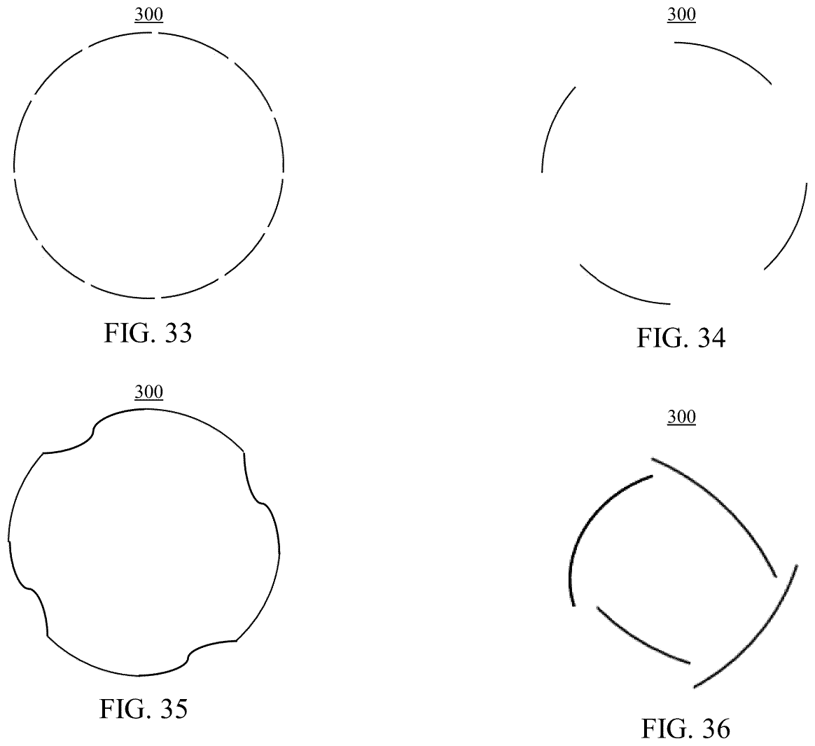

FIGS. 33, 34 and 36 illustrate a cross-section of the frame 300 or a frame section 350 that is formed of a plurality of arcuate pieces. Two or more or the arcuate pieces can have the same or different shape, size and/or configuration, and/or be formed of the same or different material. The arcuate pieces illustrated in FIGS. 33 and 34 are configured to not overlap one another when the expandable sheath 100 is in the unexpanded position and expanded position. The arcuate pieces illustrated in FIG. 36 are configured to overlap one another when the expandable sheath 100 is in the unexpanded position and optionally not overlap when the expandable sheath 100 is in the expanded position. The spacing of the one or more arcuate pieces from one another allows for the expansion of the expandable sheath 100. When the one or more arcuate pieces are formed of a shape memory material, such one or more arcuate pieces can be used to facilitate in causing the expandable sheath 100 to move back to the unexpanded position after being expanded. In such an arrangement, the expansion of the expandable sheath 100 causes one or more of the arcuate pieces to bend. The shape memory properties of the material used to partially or fully form the arcuate pieces that have been bent will cause the arcuate piece to reform back to the position prior to being bent, which in turn will facilitate in causing the expandable sheath 100 to move back to the unexpanded position after being expanded.

FIG. 35 illustrates a cross-section of the frame 300 or a frame section 350 that is formed of a single piece form of a plurality of arcuate portions. The arcuate shape the arcuate portions of the frame 300 or a frame section 350 allows for the expansion of the expandable sheath 100. When the one or more arcuate portions are formed of a shape memory material, such one or more arcuate portions can be used to facilitate in causing the expandable sheath 100 to move back to the unexpanded position after being expanded. In such an arrangement, the expansion of the expandable sheath 100 causes one or more of the arcuate portions to bend. The shape memory properties of the material used to partially or fully form the arcuate portions that have been bent will cause the arcuate portions to reform back to the position prior to being bent, which in turn will facilitate in causing the expandable sheath 100 to move back to the unexpanded position after being expanded.

In one non-limiting manufacturing process, after the expandable sheath 100 is formed in the fully expanded orientation (e.g., formed by extrusion, co-extrusion, stamping, molding, etching, etc.), the expandable sheath 100 can be formed into the final fully contracted orientation by causing the flexible portions or thin bands 1200 to be bent or folded and/or optionally twisted. The arrangement used to bend or fold and/or optionally twist the flexible portions or thin bands 1200 is non-limiting. As illustrated in FIGS. 23, 27, 29 and 30, the flexible portions or thin bands 1200 are bent at about one third the circumferential length of the flexible portions or thin bands 1200 to form a bent flexible portions or thin bands 1200 having three stacked layers. As can be appreciated, the flexible portions or thin bands 1200 can be bent to form more than three stacked layers (e.g., 5 layers, 7 layers, etc.). The length of each of the overlapping layers of the one or more flexible portions or thin bands 1200 can be the same or substantially the same as illustrated in FIGS. 23, 27, 29 and 30; however, this is not required. As illustrated in FIGS. 23, 27, 29 and 30, the flexible portions or thin bands 1200 have a generally S-shaped or serpentine or sinuous configuration when the expandable sheath 100 in the fully contracted orientation. When the expandable sheath 100 is expanded to the fully expanded orientation as illustrated in FIGS. 21, 24 and 25, the one or more flexible portions or thin bands 1200 are caused to be reshaped from the S-shape or serpentine or sinuous configuration to an arc-shape configuration. In the fully expanded configuration, one or more of the flexible portions or thin bands 1200 do not have any overlapping portion as illustrated in FIGS. 21, 24 and 25.

As illustrated in FIGS. 23, 27, 29 and 30, the two rounded ends of the three stack or overlapping layers of the flexible portions or thin bands 1200 are being positioned adjacent to a tapered region 1230 of flexible portions or thin bands 1200. As illustrated in FIGS. 232, 27, 29 and 30, the folded flexible portions or thin bands 1200 are equally displaced from each other around the circumference of the expandable sheath 100; however, this is not required.

Generally, the flexible portions or thin bands 1200 are partially or fully formed of a shape memory material. The generally rigid structures or thick bands 1300 may or may not be partially or fully formed of a shape memory material.

The flexible portions or thin bands 1200 can optionally be folded prior to being fully cooled (when the polymer is heated during the extrusion of the one or more polymers when forming the expandable sheath) and/or the folded flexible portions or thin bands 1200 can be optionally heated and then cooled while in a folded state so as to create some memory in the flexible portions or thin bands 1200 so that the flexible portions or thin bands 1200 want to maintain its folded or partially folded state after the expandable sheath 100 has been expanded from its fully contracted orientation. The heat treatment process may vary when the shape memory material is a polymer or a metal alloy. Such a shape memory feature can be advantageous 1) maintaining the medical device in a non-expanded orientation during the deployment of a medical device in a patient; 2) causing the expandable sheath 100 to partially or fully return to its fully contracted orientation after expansion be a medical device being passed through the expandable sheath 100; and/or 3) causing the expandable sheath 100 to partially or fully return to its fully contracted orientation to facilitate in the removal of the expandable sheath 100 from a patient after the medical device has been inserted into the patient.

One or more outer surfaces of the flexible portions or thin bands 1200 and/or generally rigid structures or thick bands 1300 can optionally include a lubrication coating and/or liner to facilitate in the movement of a medical device through the expandable sheath 100. In one non-limiting embodiment, the surfaces of the flexible portions or thin bands 1200 and/or generally rigid structures or thick bands 1300 are coated with a lubrication coating and/or includes a liner has a coefficient of friction of no more about 0.1 (e.g., 0.0001-0.1 and all values and ranges therebetween). Non-limiting examples of lubrication materials include PTFE, polyethylene, polyvinylidene fluoride, and combinations thereof. In one non-limiting configuration, the inner surface of the inner layer portion includes a coating of PTFE.

One or more outer surfaces of the flexible portions or thin bands 1200 and/or generally rigid structures or thick bands 1300 can optionally include a hydrophilic coating to facilitate in the insertion of the expandable sheath 100 into and/or out of a body passageway. Non-limiting examples of hydrophilic coatings include the Harmony™ Advanced Lubricity Coatings and other Advanced Hydrophilic Coatings available from SurModics, Inc., Eden Prairie, Minn. DSM medical coatings (available from Koninklijke DSM N.V, Heerlen, the Netherlands).

The expandable sheath 100 can optionally include one or more radiopaque markers or fillers. The radiopaque filler or marker, when used, can be located on the outer surface of the expandable sheath, embedded on one or more layers of the expandable sheath, and/or be located between on or more layers of the expandable sheath. The location of the one or more radiopaque markers or fillers on the expandable sheath 100 is non-limiting. Non-limiting materials that can be used as a radiopaque filler or marker include barium sulfite, bismuth trioxide, titanium dioxide, and/or bismuth subcarbonate.

The expandable sheath 100 in accordance with the present disclosure can be used a) to minimize trauma to a body passageway (e.g., blood vessel, etc.) by allowing for temporary expansion of a portion of the expandable sheath to accommodate a medical device and/or a delivery system for a medical device, and thereafter the expandable sheath is configured to return to its original diameter or close to its original diameter once the medical device and/or a portion of the delivery system passes through expandable sheath 100, b) to reduce the length of time a procedure takes, c) to reduce the risk of a longitudinal or radial body passageway tear, d) to reduce risk of plaque dislodgement in a body passageway, e) to reduce or eliminate the need for multiple insertions sheaths or other devices for the dilation of a body passageway, f) for many types of minimally invasive surgery, such as any surgery requiring introduction of a medical device (e.g., stent, prosthetic heart valve, grafts, etc.) into a body passageway (e.g., veins, arteries, esophagus, ducts of the biliary tree, intestine, urethra, fallopian tube, other endocrine or exocrine ducts, etc.).

The expandable sheath 100, in accordance with the present disclosure, can be used with various methods of introducing a medical device into a patient's vasculature. One such method comprises positioning an expandable sheath in a patient's vessel, passing a medical device through the expandable sheath 100, which causes a portion of the expandable sheath surrounding the device to expand and accommodate the profile of the medical device, and then retracting the expanded portion of the expandable sheath 100 to its original size or near original size after the medical device has partially or fully passed through the expandable sheath 100. In some methods, the expandable sheath 100 can be sutured to the patient's skin at the insertion site so that once the expandable sheath 100 is inserted the proper distance within the patient's vasculature, it does not move once the implantable medical device starts to travel through the expandable sheath 100.

The expandable sheath 100 in accordance with the present disclosure can be used with other delivery and minimally invasive surgical components, such as an introducer and loader. In one non-limiting embodiment, the expandable sheath 100 can be flushed to purge any air within the expandable sheath 100. An introducer can be inserted into the expandable sheath 100 and the introducer/sheath combination can be fully inserted into vasculature over a guiding device, such as a guidewire. Once the expandable sheath 100 and introducer are fully inserted into a patient's vasculature, the expandable sheath 100 can be optionally sutured in place at the insertion site. In this manner, the expandable sheath 100 can be substantially prevented from moving once positioned within the patient. The introducer can then be removed and a medical device, such as a transcatheter heart valve can be inserted through the expandable sheath 100. Such methods can additionally comprise placing the heart valve in a crimped state on the distal end portion of an elongated delivery apparatus, and inserting the elongated delivery device with the crimped valve into and through the expandable sheath 100. Next, the delivery apparatus can be advanced through the patient's vasculature to the treatment site, where the valve can be implanted. Typically, the medical device has a greater outer diameter than the diameter of the expandable sheath 100 in its fully contracted orientation. The medical device can be advanced through the expandable sheath 100 towards the implantation site, and the expandable sheath 100 can locally expand to accommodate the medical device as the device passes through the expandable sheath 100. The radial force exerted by the medical device on the interior passageway or cavity of the expandable sheath 100 can be sufficient to locally expand the expandable sheath 100 to an expanded diameter (e.g., the fully expanded orientation) just in the area where the medical device is currently located or the complete sheath can be caused to be expanded along its longitudinal length. Once the medical device passes a particular location of the expandable sheath 100, the expandable sheath 100 can at least partially contract to a smaller diameter. Alternatively, the expandable sheath 100 can be configured to contract after the medical device has fully passed through the expandable sheath 100. Once the medical device is implanted, the expandable sheath 100 and any sutures holding it in place can be removed.

Expandable sheath 100 can be configured to expand to an expanded outer diameter that is from about 10% greater than the original unexpanded outer diameter to about 500% greater than the original unexpanded outer diameter.

Although example embodiments have been disclosed, a worker of ordinary skill in this art would recognize that certain modifications would come within the scope of the claims. For that reason, the following claims should be studied to determine their true scope and content.

It will thus be seen that the objects set forth above, among those made apparent from the preceding description, are efficiently attained, and since certain changes may be made in the constructions set forth without departing from the spirit and scope of the disclosure, it is intended that all matter contained in the above description and shown in the accompanying drawings shall be interpreted as illustrative and not in a limiting sense. The disclosure has been described with reference to preferred and alternate embodiments. Modifications and alterations will become apparent to those skilled in the art upon reading and understanding the detailed discussion of the disclosure provided herein. This disclosure is intended to include all such modifications and alterations insofar as they come within the scope of the present disclosure. It is also to be understood that the following claims are intended to cover all of the generic and specific features of the disclosure herein described and all statements of the scope of the disclosure which, as a matter of language, might be said to fall therebetween. The disclosure has been described with reference to the preferred embodiments. These and other modifications of the preferred embodiments, as well as other embodiments of the disclosure, will be obvious from the disclosure herein, whereby the foregoing descriptive matter is to be interpreted merely as illustrative of the disclosure and not as a limitation. It is intended to include all such modifications and alterations insofar as they come within the scope of the appended claims.

To aid the Patent Office and any readers of this application and any resulting patent in interpreting the claims appended hereto, applicants do not intend any of the appended claims or claim elements to invoke 35 U.S.C. 112 (f) unless the words “means for” or “step for” are explicitly used in the particular claim.

What is claimed:

1. An expandable sheath that provides access to a body passageway to facilitate in delivering a medical device into the body passageway; said expandable sheath is expandable from a first cross-sectional area to a second cross-sectional area; said second cross-sectional area is greater than said first cross-sectional area; said expandable sheath includes an internal cavity that extends at least 50% of a longitudinal length of said expandable sheath; said expandable sheath includes a plurality of thin bands and a plurality of thick bands that extend along a majority or all of said longitudinal length of said expandable sheath; each of said thin bands include first and second ends; each of said thick bands includes first and second ends; said first end of each of said thick bands is connected to said second end of one of said thin bands; said second end of each of said thick bands is connected to a first end of each of said thin bands; each of said thin bands is configured to increase in longitudinal length when said expandable sheath expands from said first cross-sectional area to said second cross-sectional area; each of said thick bands is configured to have substantially a same shape and longitudinal length when said expandable sheath is expanded from said first cross-sectional area to said second cross-sectional area; each of said thin bands is configured to have a different shape when said expandable sheath is expanded from said first cross-sectional area to said second cross-sectional area; an outer perimeter of at least one of said thin bands and an outer perimeter of at least one of said thick bands has a same radius of curvature along 70-100% of said outer perimeter of said thick and thin bands when said expandable sheath is expanded to said second cross-sectional area; said thick bands are non-circumferentially continuous about an outer perimeter of said expandable sheath; said thin bands are non-circumferentially continuous about an outer perimeter of said expandable sheath; and
wherein said expandable sheath is configured to expand to said second cross-sectional area during movement of the medical device through said internal cavity of said expandable sheath; and
wherein said expandable sheath is configured to contract from said second cross-sectional area after the medical device partially or fully passes through said internal cavity of said expandable sheath.

2. The expandable sheath as defined in claim 1, wherein at least a portion of each of said thick bands and said thin bands is formed of a polymer material; said polymer material that is used to form each of said thick band has a greater durometer than said polymer material that is used to form each of said thin bands; each of said thin bands at least partially formed of a flexible or elastomeric polymer; and wherein said lower durometer of said thin bands facilitates in causing said expandable sheath to contract from said second cross-sectional area to or close to said first cross-sectional after the medical device partially or fully passes through said internal cavity of said expandable sheath.

3. The expandable sheath as defined in claim 1, wherein one or more of said thin bands is formed of a different material from one or more of said thick bands.

4. The expandable sheath as defined in claim 1, wherein a) one or more of said thin bands has an S-shaped configuration when said expandable sheath in said first cross-sectional area, and wherein one or more of said thin bands is reshaped from said S-shape configuration to an arc-shape when said expandable sheath expands from said first cross-sectional area to said second cross-sectional, or b) one or more of said thin bands have overlapping portions when said expandable sheath in said first cross-sectional area, and wherein one or more of said thin bands is reshaped from being in an overlapping configuration to a non-overlapping configuration when said expandable sheath expands from said first cross-sectional area to said second cross-sectional area.

5. The expandable sheath as defined in claim 1, including a tapered transition having a variable thickness; said tapered transition is located at an end portion of one or more of said thin bands and terminates at said first or second end of said thick band.

6. The expandable sheath as defined in claim 1, wherein a circumferential length of said one or more of said thin bands is equal to or greater than a circumferential length of said one or more of said thick bands when said expandable sheath is in said second cross-sectional area.

7. The expandable sheath as defined in claim 1, wherein one or more of said thin bands and/or one or more of said thick bands is fully formed of a polymer material.

8. The expandable sheath as defined in claim 1, wherein one or more of said thin bands and/or one or more of said thick bands is partially or fully formed of a metal material.

9. The expandable sheath as defined in claim 1, wherein one or more of said thin bands and/or one or more of said thick bands is partially or fully formed of a shape memory material that is partially or fully encapsulated in a non-shape memory material; and wherein said shape memory material one or more of said thick and/or thin bands facilitates in causing said expandable sheath to contract from said second cross-sectional area to or close to said first cross-sectional after the medical device partially or fully passes through said internal cavity of said expandable sheath.

10. The expandable sheath as defined in claim 1, wherein a) a plurality or all of said thick bands have the same shape, size, configuration and/or composition, and/or b) a plurality or all of said thin bands have the same shape, size, configuration and/or composition; said expandable sheath includes at least two thick bands and at least two thin bands.

11. The expandable sheath as defined in claim 1, wherein a thinnest thickness of one or more said thin bands is 10-80% of a thickness of a maximum thickness of one or more of said thick bands.

12. The expandable sheath as defined in claim 1, further including an expandable cover that fully encircles said thick bands and said thin bands when said expandable sheath is in said first cross-sectional area and said second cross-sectional area; said expandable cover extends 20-100% along a longitudinal length of said expandable sheath.

13. The expandable sheath as defined in claim 1, further including one or more anti-kinking members; said one or more anti-kinking members extends 20-100% along a longitudinal length of said expandable sheath.

14. The expandable sheath as defined in claim 13, wherein said one or more anti-kinking members is partially or fully formed of a shape memory material.

15. The expandable sheath as defined in claim 13, wherein first and second anti-kinking members are spaced apart from one another about a perimeter of said expandable sheath; each of said first and second anti-kinking members are formed of one or more pieces; each of said first and second anti-kinking members extends 30-100% along a longitudinal length of said expandable sheath.

16. The expandable sheath as defined in claim 1, further including a frame; said frame extends 5-100% along a longitudinal length of said expandable sheath; said frame is partially or fully encapsulated in a) one or more of said thick bands and/or b) one or more of said thin bands; said frame is configured to cause said expandable sheath to contract from said second cross-sectional area to or close to said first cross-sectional area after said expandable sheath has been expanded to said second cross-sectional area.

17. The expandable sheath as defined in claim 16, wherein said frame is partially or fully formed of a shape memory material; said shape memory material in said frame facilitates in causing said expandable sheath to contract from said second cross-sectional area to or close to said first cross-sectional after the medical device partially or fully passes through said internal cavity of said expandable sheath.

18. The expandable sheath as defined in claim 16, wherein said frame is a) formed of a single piece that extend about 20-100% of an outer perimeter of said expandable sheath when said expandable sheath is said second cross-sectional area, or b) formed of a plurality of pieces that extend about 20-100% of an outer perimeter of said expandable sheath when said expandable sheath is said second cross-sectional area; said frame is i) formed of a single piece of material that extends along 30-100% of said longitudinal length of said expandable sheath, or b) formed of a plurality of frame sections that are spaced apart from one another, and the total sum of the frame sections extends along 5-99% of said longitudinal length of said expandable sheath; said frame includes A) one or more arcuate portions, and/or B) one or more overlapping portions.

19. The expandable sheath as defined in claim 18, wherein said frame includes said plurality of frame sections that are spaced apart from one another; two or more of said frame sections are connected together by one or more spine members.

20. The expandable sheath as defined in claim 1, further including one or more breakable connection points; said one or more breakable connection points are configured to maintain one or more portions of said expandable sheath in said first cross-sectional area until the medical device passes through at least a portion of said internal cavity of said expandable sheath and causes said expandable sheath to expand to said second cross-sectional area and to thereby cause one or more of said breakable connection points to break.

21. The expandable sheath as defined in claim 20, wherein said one or more breakable connection portions extend 5-100% of a longitudinal length of said expandable sheath.

22. The expandable sheath as defined in claim 20, wherein said one or more breakable connection portions are formed of an adhesive and/or a melted connection.

23. The expandable sheath as defined in claim 1, wherein a thickness of each of said thick bands along a longitudinal length of said thick band is greater than a thickness of an articulating foldable or overlapping portion of each of said thin bands along a longitudinal length of said articulating foldable or overlapping portion of said thin band.

24. The expandable sheath as defined in claim 1, wherein a) a thickness of a foldable or overlapping portion of each of said thin bands along a longitudinal length of said thin band generally does not change when said expandable sheath is expanded from said first cross-sectional area to said second cross-sectional area, and/or b) a thickness of each of said thick bands along a longitudinal length of said thick band generally does not change when said expandable sheath is expanded from said first cross-sectional area to said second cross-sectional area.

25. The expandable sheath as defined in claim 1, further including a hydrophilic coating to facilitate in the insertion of said expandable sheath into and/or out of a body passageway.

26. The expandable sheath as defined in claim 1, further including a lubricating coating to facilitate movement of the medical device through said internal cavity of said expandable sheath.

27. A sheath that is adapted for use in the delivery of a medical device into a body passageway; said sheath is expandable and contractable between an expanded position and an unexpanded position; said sheath includes an internal cavity that extends 50-100% a longitudinal length of said sheath; said sheath comprising:
an outer layer portion; said outer layer portion is at least partially formed of a first polymer material;
an inner layer portion; said inner layer portion is at least partially formed of a second polymer material; and
a frame; said frame is at least partially positioned between said inner and outer layer portions; said frame includes first and second frame wires that extend along a 20-100% of said longitudinal length of said sheath; said first and said frame wires are spaced from one another along 20-100% of said longitudinal length of said sheath; each of said first and second frame wires includes a plurality of arcuate portions; a plurality of said arcuate portions overlap one another along a longitudinal axis of said frame when said sheath is in said unexpanded position;
and
wherein one or both of said inner layer portion and said outer layer portion forms a continuous layer about a perimeter of said sheath along 20-100% of said longitudinal length of said sheath when said sheath is in both said expanded and unexpanded positions, and
wherein said sheath is configured to expand from said unexpanded position to said expanded position as the medical device is moved through said sheath, and thereafter said sheath partially or fully returns to said unexpanded position once the medical device has passed partially or fully through said internal cavity or passageway of said sheath.

28. The sheath as defined in claim 27, wherein said outer layer portion includes one or more polymer layers.

29. The sheath as defined in claim 27, wherein said frame includes a shape memory material; said shape memory material in said frame facilitates in causing said sheath to contract from said expanded position to or close to said unexpanded position after the medical device partially or fully passes through said internal cavity of said expandable sheath.

30. The sheath as defined in claim 27, wherein said frame is substantially fully positioned between said inner and outer layer portions.

31. The sheath as defined in claim 27, wherein said first and second frame wires have substantially a same shape; said first and second wires are spaced from one another along 80-100% of said longitudinal length of said sheath.

32. The sheath as defined in claim 27, further including an expandable cover that fully encircles said outer layer portion when said expandable sheath is in both said expanded position and said unexpanded position; said expandable cover extends 20-100% along a longitudinal length of said sheath.

33. The sheath as defined in claim 27, further including one or more anti-kinking members; said one or more anti-kinking members extends 20-100% along a longitudinal length of said sheath; said one or more anti-kinking members is at least partially positioned in or between said inner layer portion and/or said outer layer portion.

34. The sheath as defined in claim 33, wherein said one or more anti-kinking members is partially or fully formed of a shape memory material.

35. The sheath as defined in claim 33, wherein first and second anti-kinking members are spaced apart from one another about a perimeter of said sheath; each of said first and second anti-kinking members are formed of one or more pieces; each of said first and second anti-kinking members extends 20-100% along a longitudinal length of said sheath.

36. The sheath as defined in claim 27, further including one or more breakable connection points; said one or more breakable connection points are configured to maintain one or more portions of said sheath in said unexpanded position until the medical device passes at least partially through said internal cavity of said sheath and causes said sheath to expand to said expanded position and to thereby cause one or more of said breakable connection points to break.

37. The sheath as defined in claim 36, wherein said one or more breakable connection portions extend 5-100% of a longitudinal length of said sheath.

38. The sheath as defined in claim 36, wherein said one or more breakable connection portions are formed of an adhesive and/or a melted connection.

39. The sheath as defined in claim 27, wherein a) a first end of said sheath is subjected to a reflow process to connect together said inner and outer layer portions and/or b) a second end of said sheath is subjected to a reflow process to connect together said inner and outer layer portions, and wherein in no more than 50% of said longitudinal length of said sheath is not subjected to said reflow process.

40. The sheath as defined in claim 27 further including a hydrophilic coating to facilitate in the insertion of said sheath into and/or out of a body passageway.

41. The sheath as defined in claim 27, further including a lubricating coating to facilitate movement of the medical device through said internal cavity of said expandable sheath.

42. A method of introducing a prosthetic heart valve into a patient's vasculature; said method comprising:
inserting an expandable sheath at least partially into a blood vessel of a patient; said expandable sheath is expandable from a first cross-sectional area to a second cross-sectional area; said second cross-sectional area is greater than said first cross-sectional area; said expandable sheath includes an internal cavity that extends at least 50% of a longitudinal length of said expandable sheath; said expandable sheath includes a plurality of thin bands and a plurality of thick bands that extend along a majority or all of said longitudinal length of said expandable sheath; each of said thin bands include first and second ends; each of said thick bands includes first and second ends; said first end of each of said thick bands is connected to said second end of one of said thin bands; said second end of each of said thick bands is connected to a first end of each of said thin bands; each of said thin bands is configured to increase in longitudinal length when said expandable sheath expands from said first cross-sectional area to said second cross-sectional area; each of said thick bands is configured to have substantially a same shape and longitudinal length when said expandable sheath is expanded from said first cross-sectional area to said second cross-sectional area; each of said thin bands is configured to have a different shape when said expandable sheath is expanded from said first cross-sectional area to said second cross-sectional area; an outer perimeter of at least one of said thin bands and an outer perimeter of at least one of said thick bands has a same radius of curvature along 70-100% of said outer perimeter of said thick and thin bands when said expandable sheath is expanded to said second cross-sectional area; said thick bands are non-circumferentially continuous about an outer perimeter of said expandable sheath; said thin bands are non-circumferentially continuous about an outer perimeter of said expandable sheath; said expandable sheath is configured to expand to said second cross-sectional area during movement of the medical device through said internal cavity of said expandable sheath; said expandable sheath is configured to contract from said second cross-sectional area after the medical device partially or fully passes through said internal cavity of said expandable sheath; and
advancing a prosthetic heart valve through said internal cavity of said expandable sheath so as to cause said expandable sheath to expand from said first cross-sectional area to said second cross-sectional as said prosthetic heart valve partially or fully passes through said internal cavity of said expandable sheath; and
wherein said expandable sheath contracts from said second cross-sectional area toward said first cross-sectional after said prosthetic heart valve has partially or fully passed through said internal cavity of said expandable sheath.

43. The method as defined in claim 42, wherein said prosthetic heart valve is a frame mounted heart valve mounted in a radially crimped state on a delivery apparatus.

44. A method of introducing a medical device into a body passageway of a patient; said method comprising:
providing a sheath; said sheath is expandable and contractable between an expanded position and an unexpanded position; said sheath includes an internal cavity that extends 50-100% a longitudinal length of said sheath; said sheath comprising:
an outer layer portion; said outer layer portion is at least partially formed of a first polymer material;
an inner layer portion; said inner layer portion is at least partially formed of a second polymer material; and
a frame; said frame is at least partially positioned between said inner and outer layer portions; said frame includes first and second frame wires that extend along a 20-100% of said longitudinal length of said sheath; said first and said frame wires are spaced from one another along 20-100% of said longitudinal length of said sheath; each of said first and second frame wires includes a plurality of arcuate portions; a plurality of said arcuate portions overlap one another along a longitudinal axis of said frame when said sheath is in said unexpanded position; and
wherein one or both of said inner layer portion and said outer layer portion forms a continuous layer about a perimeter of said sheath along 20-100% of said longitudinal length of said sheath when said sheath is in both said expanded and unexpanded positions, and
wherein said sheath is configured to expand from said unexpanded position to said expanded position as the medical device is moved through said sheath, and thereafter said sheath partially or fully returns to said unexpanded position once the medical device has passed partially or fully through said internal cavity or passageway of said sheath;
b. inserting said sheath at least partially into said body passageway; and
c. advancing said medical device through said expandable sheath to cause said expandable sheath to locally expand from said unexpanded position to said expanded position due to movement of said medical device through said expandable sheath; and
wherein said expandable sheath partially or fully contracts back to said unexpanded position after said medical device has partially or fully passed through said expandable sheath.

45. The method as defined in claim 44, wherein said step of inserting includes passing said expandable sheath transcutaneously through a surgically-created opening in the patient's skin such that at least a portion of said outer portion of said sheath is positioned adjacent to said surgically-created opening.

46. The method as defined in claim 44, wherein said medical device in a prosthetic heart valve mounted in a radially crimped state on a delivery apparatus.
